(19) United States
(12) Patent Application Publication
(10) Pub. No.: US 2011/0195048 A1
(43) Pub. Date: Aug. 11, 2011

(54) REGULATION OF LYMPHOCYTES AND USES THEREFOR

(76) Inventor: Eckhard R. Podack, Coconut Grove, FL (US)

(21) Appl. No.: 13/123,280

(22) PCT Filed: Oct. 8, 2009
(86) PCT No.: PCT/US09/59966

§ 371 Date: Apr. 26, 2011

Related U.S. Application Data
(60) Provisional application No. 61/103,813, filed on Oct. 8, 2008.

(51) Int. Cl. A61K 38/20; A61K 39/395; A61K 39/00; C12N 5/0783; C07H 21/00; C07K 14/00; C07K 16/28; A61P 37/02

(52) U.S. Cl. 424/85.2; 424/158.1; 424/184.1; 435/325; 536/23.5; 530/351; 530/389.1

(57) ABSTRACT

Compositions comprising TNF receptor super-family member 25 (TNFR25) agents, attenuate Treg activity and, by comparison with other TNFR members, only weakly costimulates T effector cell (Teff) activity. Alternatively spliced TNFR25 modulates the functional effects of TNFR25 signaling These agents have a wide therapeutic applicability in the treatment of diseases by modulating immune responses. In addition these agents can be used in conjunction with vaccines to enhance the immune response.

### CROSS-REFERENCE TO RELATED APPLICATIONS

The present application claims the priority of U.S. provisional patent application No. 61/103,813 filed Oct. 8, 2008, which is incorporated herein by reference in its entirety.

### FIELD OF THE INVENTION

Embodiments are related to TNF receptor super-family member 25 (TNFR25) molecules for regulation of an immune response and for polarization of a T helper lymphocyte type 1 or type 2 mediated response.

### BACKGROUND

TNF receptor super-family member 25 (TNFR25), also known as Wsl-1, Apo3, LARD, TRAMP, DR3, TR3 (Bodmer, J. L., et al., 1997. Immunity 6:79-88; Chinnaiyan, A. M., et al., 1996. Science 274:990-992; Kitson, J., et al. 1996. Nature 384:372-375; Marsters, S. A., et al., 1998. Curr Biol 8:525-528; Screaton, G. R., et al. 1997. Proc Natl Acad Sci USA 94:4615-46191-5), is a member of the TNF-receptor family with a typical death domain. Like other death receptors, TNFR25 can signal either for caspase activation and cell death, or promote cell survival through the NF-κB and MAPK cascade. TNFR25 is expressed primarily in lymphoid cells in the form of randomly spliced transcripts. After lymphocyte activation TNFR25 protein expression is up-regulated and the full-length isoform of TNFR25 becomes predominant.

TNFR25 and its ligand TL1A (TNFSF15) appear to have important functions in the pathogenesis of colitis, in atopic lung inflammation—a model for asthma, in experimental allergic encephalitis—a model for multiple sclerosis and in rheumatoid arthritis. The diverse functions of TNFR25/TL1A have been attributed mainly to a costimulatory role for Th1 cytokine production (Migone, T. S., et al. 2002. Immunity 16:479-492; Bamias, G., et al. 2006. Proc Natl Acad Sci USA 103:8441-8446; Papadakis, K. A., et al. 2004. J Immunol 172:7002-7007) and, more recently, for Th17 polarization and IL-17 production (Pappu, B. P., et al. 2008. J Exp Med 205:1049-1062).

Signaling through TNFR members in CD4 lymphocytes has synergistic effects by costimulating effector cells while simultaneously inhibiting regulator cells that otherwise would attenuate immune responses. GITR was the first TNFR family member found to be expressed by Tregs. Triggering of GITR with an agonistic antibody abrogated CD4⁻CD25⁺ Treg cell-mediated suppression and induced autoimmune gastritis in recipient animals (Shimizu, J., S. et al., 2002. Nat Immunol 3:135-142). GITR also was costimulatory for naïve T cells and for Th1 and Th2 polarized T cells (Tone, M., et al. 2003. Proc Natl Acad Sci USA 100:15059-15064), and rendered CD25-cells more resistant to Treg suppressive activity (Stephens, G. L., et al. 2004. J Immunol 173:5008-5020). Administration of anti-GITR antibodies exacerbated mouse allergic pulmonary inflammation and EAE and augmented anti-tumor (Ko, K., et al., 2005. J Exp Med 202:885-891) and anti-viral immune responses (Chen, M. L., et al. 2005. Proc Natl Acad Sci USA 102:419-424). GITR-deficient mice are more resistant to collagen-induced arthritis, since GITR^{-/-} Tregs display higher regulatory activity (Cuzzocrea, S., et al. 2005. Faseb J 19:1253-1265).

### SUMMARY

This Summary is provided to present a summary of the invention to briefly indicate the nature and substance of the invention. It is submitted with the understanding that it will not be used to interpret or limit the scope or meaning of the claims.

Embodiments of the invention are directed to compositions which modulate the immune response. The compositions comprise TNFR25, fragments, variants, mutants, alleles, splice variants, isoforms, analogs or combinations thereof. Examples of splice variants, include a natural splice form of TNFR25, e.g. full length (FL) TNFR25 and Δ5,6-TNFR25, the splice form lacking exon 5 and 6 of the extracellular domain but retaining the transmembrane and signaling domain. TNFR25 signals suppress the activity of T regulatory cells and increase the frequency of CD4⁺FoxP3⁺CD25⁻ cells. In CD4 effector cells TNFR25 signals enhance the production of IL-17 and TH2-cytokines, which in turn upregulate TL1A on effector cells. Compared to FL-TNFR25, the effects of Δ5,6-TNFR25 signaling are attenuated, allowing fine tuning of the immune response.

Other aspects of the invention are described infra.

### BRIEF DESCRIPTION OF THE DRAWINGS

FIGS. 1A-1C: Full length TNFR25 expression by activation induced splicing. FIG. 1A is a schematic representation showing splice forms of murine TNFR25 mRNA obtained from resting spleen cells by sequencing of reverse transcribed cDNA, PCR amplification and cloning. FL—full length TNFR25; splice form 1-4 contain the unspliced 2^{nd} intron between exon 2 and 3; the asterisk denotes an in-frame stop codon. Δ followed by numerals indicates missing exon(s). CRD I-IV—cysteine rich domains characteristic for the TNF-receptor family. TM—trans-membrane domain (blue when present in predicted protein); DD—death domain (red when present in predicted protein). FIG. 1B is a blot showing the kinetics of human TNFR25 splicing by PMA and ionomycin. Four splice variants are resolved and labeled. Human peripheral blood mononuclear cells were activated with PMA (10 ng/ml) and ionomycin (400 ng/ml) for the time indicated. FIG. 1C is a blot showing activation induced splicing of human TNFR25 mRNA is inhibited by protein kinase C inhibitor H7 and is not dependent on protein synthesis. Human PBMC were activated with PMA (10 ng/ml) and ionomycin (400 ng/ml) for 12 hours. Where indicated, the cells were pretreated with cycloheximide (10 μg/ml) or H7 (50 μM) for 30 min before adding PMA and ionomycin.

FIGS. 2A-2C: Diminished cellularity and T cell proliferation of FL- and Δ5,6-TNFR25 transgenic cells. FIG. 2A is a graph showing that cellularity is reduced in TNFR25 transgenic mice. Single cell suspensions of thymus, spleen, and inguinal lymph nodes were analyzed. FIG. 2B is a graph showing that T lymphocyte frequency is reduced in TNFR25 transgenic mice. Cells were stained with CD4-Cychrome or CD8-PE to determine the percentage of CD4 or CD8 T cells by flow cytometry. Statistical calculations were carried out using littermates as controls. n.s.: not significant; *p<0.05; **p<0.01; ***p<0.001. FIG. 2C is a graph showing impaired activation-induced proliferation in TNFR25 transgenic mice. Proliferation of splenocytes upon stimulation for 3 days was measured by 3H-thymidine incorporation. Splenocytes or purified T cells were activated with immobilized anti-mouse CD3 (2C11, 2 μg/ml) with or without soluble anti-mCD28 (1 μg/ml), or with Concanavalin A (5 μg/ml), or PMA (10 ng/ml) and ionomycin (400 ng/ml). Recombinant mouse IL-2 was used where indicated at 1000 U/ml.

FIGS. 3A-3E: TNFR25 transgenic CD4 cells spontaneously produce high levels of Th2 and Th17 cytokines. FIG. 3A is a series of histograms showing expression of endogenous and transgenic TNFR25. Lymph node cells were isolated and stained and TNFR25 fluorescence displayed by gating on the population indicated in the histograms. Black curve represents control hamster IgG. Green curves and digits are endogenous TNFR25 and MFI, red curves and digits are transgenic TNFR25 and MFI, black curves are isotype control. FIG. 3B is a graph showing cytokine production by TNFR25 transgenic CD4 T cells during the primary response. CD4 T cells from spleens were purified by negative selection and activated with immobilized anti-CD3 (2 μg/ml) and soluble anti-mCD28 (1 μg/ml) for 3 days. The supernatants were collected for cytokine ELISA assays. The figure is representative of three independent experiments. *p<0.05; **p<0.01; ***p<0.001. FIG. 3C is a graph showing the expression of TL1A mRNA in polarized wt CD4 T cells and in unstimulated transgenic CD4 T cells. Left panel: wt CD4 T cells were stimulated with immobilized anti-CD3 (2 μg/ml) and soluble anti-CD28 (1 μg/ml) alone for ThN polarization, or in combination with IL-12 (5 ng/ml) and anti-IL-4 (20 μg/ml) for Th1 polarizing conditions; with IL-4 (10 ng/ml), anti-IFN-γ (10 μg/ml), and anti-IL-12 (10 μg/ml) for Th2 polarization and in combination with IL-1β (10 ng/ml), TGF-β (5 ng/ml) and IL-6 (20 ng/ml) with 10 μg/ml of anti-IFN-γ and 5 μg/ml of anti-IL-4 for Th17 polarizing conditions for 4 days. After washing and re-plating with anti-CD3 for 48 h, RNA was extracted using QIAGEN's RNeasy Mini Kit followed by reverse transcription and rtPCR for TL1A using an ABI Taqman probe. Data shown are GAPDH normalized and relative to ThN polarizing conditions. Right panel: RNA was extracted from purified, un-stimulated CD4 T cells fromw.t.and transgenic mice. After reverse transcription, rtPCR was performed for TL1A. Data shown are GAPDH normalized and relative to wt. FIG. 3D is a graph showing spontaneous TH2 and enhanced TH17 polarization by TNFR25 transgenic CD4 cells. CD4 cells were activated with immobilized anti-CD3 (2 μg/ml) and soluble anti-CD28 (1 μg/ml) alone (ThN), or combined with IL-12 (5 ng/ml) and anti-IL-4 (20 μg/ml) for Th1 polarization, or combined with IL-4 (10 ng/ml), anti-IFN-γ (10 μg/ml), and anti-IL-12 (10 μg/ml) for Th2 polarization or combined with IL-1β (10 ng/ml), TGF-β (5 ng/ml) and IL-6 (20 ng/ml) with 10 μg/ml of anti-IFN-γ and 5 μg/ml of anti-IL-4 for Th17 polarization for 4 days. After washing and re-plating with anti-CD3 for 24 h, the supernatants were collected for cytokine ELISA analysis. FIG. 3E shows increased lung inflammation in Δ5,6-TNFR25 transgenic mice. Mice were sensitized intraperitoneally with 66 μg ovalbumin mixed in 6 mg alum on day 0 and day 5. On day 12, mice were aerosol challenged with 0.5% ovalbumin in PBS for one hour using an ultrasonic nebulizer. Three days after the single aerosol exposure allergic inflammation in the lung was analyzed. Left panels: Lung pathology after staining with hematoxylin-eosin (H&E) and periodic acid Schiff's stain (PAS). Right panels: The trachea was cannulated and the lung was lavaged 4 times with 1 ml of PBS. The cells recovered from bronchoalveolar lavage fluid (BALF) were counted and cytospin slides were prepared and stained with Giemsa-Wright for differential cell counts.

FIGS. 4A-4H: TNFR25 agonists inhibit Treg activity and costimulate CD4 effector cells (Teff). FIG. 4A is a cell sorter scan showing that Treg express TNFR25. Purified CD4^{|} CD25⁻ cells were expanded with anti CD3/anti C28 beads and IL-2 and analyzed by flow cytometry for expression of CD4, CD25, TNFR25 and intracellular FoxP3. In an overlay, green, upper right panel, the expression of FoxP3 in resting CD4⁺CD25⁻ cells is also shown. FIG. 4B is a graph show that agonistic 4C12 costimulates CD4 effectors and blocks Treg. Wild type CD4⁺CD25⁻ T effector cells (Teff) and CD4⁺CD25⁺ Treg were mixed at indicated ratios and activated with soluble anti-CD3 (1 μg/ml) for three days in the presence of control IgG or the agonistic anti-TNFR25 antibody 4C12 (5 μg/ml). FIG. 4C is a graph showing that agonistic TL1A costimulates CD4 effectors and blocks Treg. Details as in FIG. 4A except that instead of antibody, soluble recombinant MBP-TL1A fusion protein was added at 1 μg/ml as TNFR25 agonist. FIG. 4D is a graph showing that agonistic TL1A costimulates blocks Treg and does not costimulate DN-TNFR25 transgenic Teff. DN-TNFR25 transgenic effector cells (DN Teff) were mixed with wild type Treg and TL1A as in FIG. 4B. FIG. 4E is a graph showing that Treg from FL-TNFR25 transgenic mice do not inhibit proliferation of CD4⁺CD25⁻ cells. FL-TNFR25 CD4⁺CD25⁺ Treg were mixed with wild type CD4⁺CD25⁻ cells and antibodies as in FIG. 4A. FIG. 4F is a graph showing that Δ5,6-TNFR25 Treg are highly sensitive to TNFR25 agonists. Wild type Teff cells were mixed with Δ5,6-TNFR25 transgenic Treg and antibodies as in FIG. 4A. FIG. 4G is a graph showing that OT-I TCR transgenic CD8 effector cells are not costimulated by 4C12 but their inhibition by Treg is relieved by 4C12. TCR-transgenic, ovalbumin specific CD8 cells (OT-I) were stimulated with the indicated concentration of the cognate peptide SIINFEKL (SEQ ID NO: 1). CD4⁺CD25⁺ Treg were added at a 1:1 ratio. 4C12 agonistic anti TNFR25 antibody was at 5 μg/ml, where indicated; incubation for three days. FIG. 4H is a graph showing that FL-TNFR25 transgenic Treg do not inhibit OT-1. OT-I were mixed with wild type or FL-TNFR25 transgenic Treg at several ratios in the presence of 4C12 or control IgG (5 μg/ml) for three days and activated with SIINFEKL (SEQ ID NO: 1) peptide (10⁻¹¹ M). Data show ³H-thymidine incorporation during the final 4 hours presented as cpm±SEM of three or four replicates. ***p<0.01; *p<0.1

FIGS. 5A-5B: TNFR25 costimulatory activity compared to that of OX40 and GITR. FIG. 5A is a graph showing purified CD4⁺CD25⁻ cells (Teff) which were mixed with CD4⁺CD25⁺ Treg at indicated ratios and activated for three days with soluble anti-CD3 (1 μg/ml) and with the agonistic anti-TNFR25 antibody 4C12 (10 μg/ml) or with the agonistic anti-OX40 antibody OX86 (10 μg/ml) or with both. FIG. 5B: details are like in FIG. 5A, but 4C12 was compared to the agonistic anti-GITR antibody DTA-1 (10 μg/ml). Data show ³H-thymidine incorporation presented as cpm±SEM during the final 4-6 hours of incubation.

FIG. 6 is a schematic representation of TL1A TNFR25 action on Teff and Treg. TL1A is upregulated by TH2 and TH17 polarization. TL1A is membrane bound and can be cleaved and released into solution in active form. TNFR25 signals, elicited by TL1A, support Th2⁻ and Th17 cytokine production. At the same time TNFR25 signals, elicited by TL1A on CD4⁺ Treg cells, suppress Treg inhibition of immune response.

FIG. 7 shows that TNFR25 agonistic antibody 4C12 enhances CD8^{|} CTL expansion in vivo. Mice received 10⁶ TCR-tg gfp marked OT-I i.v. 2 days later they were immunized with 2 million 3T3-gp96-Ig-ova. 50 microgram 4C12 antibody was given on the days indicated. On day 5 OT-I-gfp-CTL expansion was determined at various locations as indicated. 4C12 caused strong increases in OT-I expansion.

FIG. 8 is a graph showing that TNFR25 agonistic antibody 4C12 causes HIV-gag specific CTL expansion. HLA A2-transgenic mice were immunized with 293-HIV-gag-gp96-Ig. 4C12 was given i.p. as indicated. On day 5 the frequency of HLA A2-gag-pentaamer specific CD8 CTL was determine at several sites. 4C12 enhance HLA A2 gag specific CTL expansion.

FIG. 9 is a graph showing that agonistic anti TNFR25 4C12 mediates increased tumor rejection in conjunction with cancer vaccine. Mice were transplanted with EG7 tumors. After 5 days were vaccinated as indicated by the arrows with 2 million EG7-gp96-Ig with or without 4C12 as indicated. Addition of 4C12 to the vaccination regiment increases tumor rejection.

### DETAILED DESCRIPTION

The present invention is described with reference to the attached figures, wherein like reference numerals are used throughout the figures to designate similar or equivalent elements. The figures are not drawn to scale and they are provided merely to illustrate the instant invention. Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events. Furthermore, not all illustrated acts or events are required to implement a methodology in accordance with the present invention.

All genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the molecules disclosed herein e.g. TNFR25, full length (FL) TNFR25 and Δ5,6-TNFR25 is not limited to mice but the human antibody is preferred, which in some embodiments relate to mammalian nucleic acid and amino acid sequences are intended to encompass homologous and/or orthologous genes and gene products from other animals including, but not limited to other mammals, fish, amphibians, reptiles, and birds. In preferred embodiments, the genes or nucleic acid sequences are human.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

### Definitions

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

A “T regulatory cell” or “Treg cell” or “Tr cell” refers to a cell that can modulate a T cell response. Treg cells express the transcription factor Foxp3, which is not upregulated upon T cell activation and discriminates Tregs from activated effector cells. Tregs are identified by the cell surface markers CD25, CTLA4, and GITR. Several Treg subsets have been identified that have the ability to inhibit autoimmune and chronic inflammatory responses and to maintain immune tolerance in tumor-bearing hosts. These subsets include interleukin 10-(IL-10-) secreting T regulatory type 1 (Tr1) cells, transforming growth factor-β-(TGF-β-) secreting T helper type 3 (Th3) cells, and “natural” CD4⁺/CD25⁻ Tregs (Trn) (Fehervari and Sakaguchi. J. Clin. Invest. 2004, 114:1209-1217; Chen et al. Science. 1994, 265: 1237-1240; Groux et al. Nature. 1997, 389: 737-742).

“TNFR25 molecules”, “TNFR25 agent”, “TNFR25 composition”, “TNFR25” are used interchangeably herein and refer to, without limitation, TNFR25, TNFR25 splice variants, TNFR25-agonists, agonistic anti TNFR25, TNFR25 antagonists, TNFR25 ligands, polynucleotides, oligonucleotides, sense and antisense polynucleotide strands, complementary sequences, polypeptides, peptides, proteins, homologous and/or orthologous TNFR25 molecules RNA, DNA, analogs, isoforms, precursors, mutants, variants, derivatives, splice variants, alleles, different species, and active fragments thereof. An agonist is a substance that binds to the TNFR25 receptor and triggers a response in the cell on which the TNFR25 receptor is expressed similar to a response that would be observed by exposing the cell to a natural TNFR25 ligand, e.g., TL1A. An antagonist is the opposite of an agonist in the sense that while an antagonist may also bind to the receptor, it fails to activate the receptor and actually completely or partially blocks it from activation by endogenous or exogenous agonists. A partial agonist activates a receptor but does not cause as much of a physiological change as does a full agonist. Alternatively, another example of a TNFR25 agonist is an antibody that is capable of binding and activating TNFR25. An example of an anti-TNFR antibody is 4C12 (agonist). (Deposited under the Budapest Treaty on Behalf of: University of Miami; Date of Receipt of seeds/strain(s) by the ATCC®: May 5, 2009; ATCC® Patent Deposit Designation: PTA-10000. Identification Reference by Depositor: Hybridoma cell line; 4C12; The deposit was tested Jun. 4, 2009 and on that date, the seeds/strain(s) were viable. International Depository Authority: American Type Culture Collection (ATCC®), Manassas, Va., USA).

“TNFR25 antagonist” is referred to herein as a substance that inhibits the normal physiological function of a TNFR25 receptor. Such agents work by interfering in the binding of endogenous receptor agonists/ligands such as TL1A, with TNFR25 receptor.

The term “induces or enhances an immune response” is meant causing a statistically measurable induction or increase in an immune response over a control sample to which the peptide, polypeptide or protein has not been administered. Preferably the induction or enhancement of the immune response results in a prophylactic or therapeutic response in a subject. Examples of immune responses are increased production of type 1 IFN, increased resistance to viral and other types of infection by alternate pathogens. The enhancement of immune responses to tumors (anti-tumor responses), or the development of vaccines to prevent tumors or eliminate existing tumors.

The term “active fragment or variant” is meant a fragment that is at least 380 amino acid residues in length and is 100% identical to a contiguous portion of the peptide, polypeptide or protein, or a variant that is at least 90%, preferably 95% identical to a fragment up to and including the full length peptide, polypeptide or protein. A variant, for example, may include conservative amino acid substitutions, as defined in the art, or nonconservative substitutions, providing that at least e.g. 10%, 25%, 50%, 75% or 90% of the activity of the original peptide, polypeptide or protein is retained. Also included are TNFR25 molecules, fragments or variants having post-translational modifications such as sumoylation, phosphorylation glycosylation, splice variants, and the like, all of which may effect the efficacy of TNFR25 function.

Unless otherwise indicated, the terms “peptide”, “polypeptide” or “protein” are used interchangeably herein, although typically they refer to peptide sequences of varying sizes.

The term “variant,” when used in the context of a polynucleotide sequence, may encompass a polynucleotide sequence related to a wild type gene. This definition may also include, for example, “allelic,” “splice,” “species,” “natural splice” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. Of particular utility in the invention are variants of wild type gene products. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

The resulting polypeptides generally will have significant amino acid identity relative to each other. A polymorphic variant is a variation in the polynucleotide sequence of a particular gene between individuals of a given species. Polymorphic variants also may encompass “single nucleotide polymorphisms” (SNPs) or single base mutations in which the polynucleotide sequence varies by one base. The presence of SNPs may be indicative of, for example, a certain population with a propensity for a disease state, that is susceptibility versus resistance.

Derivative polynucleotides include nucleic acids subjected to chemical modification, for example, replacement of hydrogen by an alkyl, acyl, or amino group. Derivatives, e.g., derivative oligonucleotides, may comprise non-naturally-occurring portions, such as altered sugar moieties or inter-sugar linkages. Exemplary among these are phosphorothioate and other sulfur containing species which are known in the art. Derivative nucleic acids may also contain labels, including radionucleotides, enzymes, fluorescent agents, chemiluminescent agents, chromogenic agents, substrates, cofactors, inhibitors, magnetic particles, and the like.

A “derivative” polypeptide or peptide is one that is modified, for example, by glycosylation, pegylation, phosphorylation, sulfation, reduction/alkylation, acylation, chemical coupling, or mild formalin treatment. A derivative may also be modified to contain a detectable label, either directly or indirectly, including, but not limited to, a radioisotope, fluorescent, and enzyme label.

The term “immunoregulatory” or “modulator” is meant a compound, composition or substance that is immunogenic (i.e. stimulates or increases an immune response) or immunosuppressive (i.e. reduces or suppresses an immune response). Thus, a TNFR25 described in embodiments herein, can immunoregulate or modulate an immune response.

TNFR25 antagonists or agonists may be in the form of aptamers. “Aptamers” are DNA or RNA molecules that have been selected from random pools based on their ability to bind other molecules. The aptamer binds specifically to a target molecule wherein the nucleic acid molecule has sequence that comprises a sequence recognized by the target molecule in its natural setting. Alternately, an aptamer can be a nucleic acid molecule that binds to a target molecule wherein the target molecule does not naturally bind to a nucleic acid. The target molecule can be any molecule of interest. For example, the aptamer can be used to bind to a ligand-binding domain of a protein, thereby preventing interaction of the naturally occurring ligand with the protein. This is a non-limiting example and those in the art will recognize that other embodiments can be readily generated using techniques generally known in the art (see, e.g., Gold et al., Annu. Rev. Biochem. 64:763, 1995; Brody and Gold, J. Biotechnol. 74:5, 2000; Sun, Curr. Opin. Mol. Ther. 2:100, 2000; Kusser, J. Biotechnol. 74:27, 2000; Hermann and Patel, Science 287:820, 2000; and Jayasena, Clinical Chem. 45:1628, 1999).

As used herein, the term “antibody” is inclusive of all species, including human and humanized antibodies and the antigenic target, for example, TNFR25, can be from any species. Thus, an antibody, for example, anti-TNFR25 can be mouse anti-human TNFR25, human anti-human TNFR25; humanized anti-human TNFR25, goat anti-human TNFR25; goat anti-mouse TNFR25; rat anti-human TNFR25; mouse anti-rat TNFR25 and the like. The combinations of antibody generated in a certain species against an antigen target, e.g. TNFR25, from another species, or in some instances the same species(for example, in autoimmune or inflammatory response) are limitless and all species are embodied in this invention. The term antibody is used in the broadest sense and includes fully assembled antibodies, monoclonal antibodies (including human, humanized or chimeric antibodies), polyclonal antibodies, multispecific antibodies (e.g., bispecific antibodies), and antibody fragments that can bind antigen (e.g., Fab′, (ab)₂, Fv, single chain antibodies, diabodies), comprising complementarity determining regions (CDRs) of the foregoing as long as they exhibit the desired biological activity.

“Target molecule” includes any macromolecule, including protein, carbohydrate, enzyme, polysaccharide, glycoprotein, receptor, antigen, antibody, growth factor; or it may be any small organic molecule including a hormone, substrate, metabolite, cofactor, inhibitor, drug, dye, nutrient, pesticide, peptide; or it may be an inorganic molecule including a metal, metal ion, metal oxide, and metal complex; it may also be an entire organism including a bacterium, virus, and single-cell eukaryote such as a protozoon.

“Treating” or “treatment” of a state, disorder or condition includes: (1) Preventing or delaying the appearance of clinical or sub-clinical symptoms of the state, disorder or condition developing in a mammal that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof; or (3) Relieving the disease, i.e., causing regression of the state, disorder or condition or at least one of its clinical or sub-clinical symptoms. The benefit to a subject to be treated is either statistically significant or at least perceptible to the patient or to the physician.

“Patient” or “subject” refers to mammals and includes human and veterinary subjects.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

A “therapeutically effective amount” or a “therapeutic amount” is an amount of a therapeutic composition sufficient to produce a measurable response (e.g., a biologically or clinically relevant response in a subject being treated).

“Sample” is used herein in its broadest sense. A sample comprising polynucleotides, polypeptides, peptides, antibodies and the like may comprise a bodily fluid; a soluble fraction of a cell preparation, or media in which cells were grown; a chromosome, an organelle, or membrane isolated or extracted from a cell; genomic DNA, RNA, or cDNA, polypeptides, or peptides in solution or bound to a substrate; a cell; a tissue; a tissue print; a fingerprint, skin or hair; and the like.

The terms “patient”, “subject” or “individual” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters; and primates.

“Diagnostic” or “diagnosed” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

### Modulation of T Regulatory Cells, Th17 and Th2 Polarization

Embodiments of the invention are directed to nucleic acid sequences and encoded products thereof, to a molecule termed herein TNFR25. The novel TNFR25 nucleic acids, polynucleotides, oligonucleotides, peptides, mutants, variants and active fragments thereof, can be used to modulate the immune response in a subject and/or for the treatment of an immune-related disorder, including treating and preventing infection by modulating immunity. Also provided are an agent reactive with the peptide, a pharmaceutical composition that includes the peptide, isolated nucleic acid molecules, an isolated nucleic acid molecules encoding the peptides, recombinant nucleic acid constructs that include the nucleic acid molecules, at least one host cell comprising the recombinant nucleic acid constructs, and a method of producing the peptide using the host cell. The present invention further provides a method for treating and/or preventing infection in a subject by administering the peptide of the invention to the subject, thereby modulating innate immunity in the subject.

TNF receptor family members which down regulate the suppressive activity of T regulatory cells include OX40 (Valzasina, B., et al. 2005. Blood 105:2845-2851; Vu, M. D., et al. 2007. Blood 110:2501-2510), 4-1BB (Choi, B. K., et al. 2004. J Leukoc Biol 75:785-791) and TNFRII (Valencia, X., et al. 2006. Blood 108:253-261). OX40 triggering abrogated the disease-preventing function of Tregs in inflammatory bowel disease (IBD) (Takeda, I., et al. 2004. J Immunol 172:3580-3589) and GVHD (Valzasina, B., et al. 2005. Blood 105:2845-2851) models.

The following is provided merely as an illustrative example and is not meat to limit or construe the invention in any way. In summary, the data described herein, show that mouse TNFR25 has several alternatively spliced mRNAs similar to human TNFR25. Two of these encoding for signaling-capable receptor isoforms and a third, artificial truncated dominant negative form of the receptor were expressed as transgenes in mice under the control of CD2 promoter, and their functional activity was studied. Low numbers and decreased proliferative capacity of TNFR25 transgenic T lymphocytes, as well as increased Th2 and Th17 cytokines production by these cells was observed. The data also indicate that TNFR25 attenuates Treg activity and, by comparison with other TNFR members, only weakly costimulates T effector cell (Teff) activity and that alternative splicing modulates the functional effects of TNFR25 signaling.

The invention contemplates modulation of an immune response (serological, cellular mucosal or otherwise) to any antigen including tumors, infectious organisms, inflammation, allergies, autoimmunity and the like.

Compositions: In a preferred embodiment, compositions comprise TNFR25, TNFR25 splice variants, TNFR25-agonists, agonistic anti TNFR25, TNFR25 antagonists, TNFR25 ligands, other immune regulating molecules, variants, mutants, fragments, or combinations thereof.

In another preferred embodiment, agonists, antagonists or ligands comprise small molecules, ligands, antibodies, aptamers, organic compounds, inorganic compounds, nucleic acids or amino acids.

In another preferred embodiment, the mammalian TNFR25, TNFR25 splice variants, TNFR25-agonists, agonistic anti TNFR25,TNFR25 antagonists, TNFR25 ligands, other immune regulating molecules, variants, mutants or fragments thereof are human molecules.

In another preferred embodiment, a composition comprises at least one of: TL1A, TL1A agonists, TL1A antagonists, TL1A ligands, other immune regulating molecules, ligands, variants, mutants or fragments thereof.

In another preferred embodiment, the TL1A, TL1A agonists, TL1A antagonists, TL1A ligands, other immune regulating molecules, ligands, variants, mutants or fragments thereof are mammalian, preferably, human molecules.

In one embodiment, the TNFR25 compositions of the present invention are targeted to the cells involved in modulation of the immune system, such as, for example, immune effector cells, cells involved in the regulation of the immune system, e.g. T regulatory cells (Treg), MSC, antigen presenting cells and the like. Examples of antigen presenting cells include, dendritic cells, B cells, monocytes/macrophages.

Peptides: The term “ TNFR25” will refer to both the nucleic acids and peptides. The invention is not limited to these two species but includes without limitation, allelic variants, species variants, splicing variants, mutants, fragments, and the like. For illustrative purposes only, these two sequences will be used throughout the specification but is not to be construed as a limitation.

In a preferred embodiment, TNFR25 peptides comprise at least five consecutive amino acid residues with the understanding that they are “active” peptides. “Active” includes one or more functions of TNFR25 which includes known functions as described herein but also any other function that is innate to the TNFR25 molecule or including one which may be altered based on any manipulation by the end user.

In another preferred embodiment, a TNFR25 peptide includes the peptide itself, chemical equivalents thereto, isomers thereof (e.g., isomers, stereoisomers, retro isomers, retro-inverso isomers, all-[D] isomers, all-[L] isomers, or mixed [L] and [D] isomers thereof), conservative substitutions therein, precursor forms thereof, endoproteolytically-processed forms thereof, such as cleavage of single amino acids from N or C terminals or immunologically active metabolites of the peptides of the invention, pharmaceutically-acceptable salts and esters thereof, and other forms resulting from post-translational modification. Also included is any parent sequence, up to and including 10, 9, 8, 7, 6, 5 and 4 amino acids in length (cyclized, or linear, or branched from the core parent sequence), for which the specified sequence is a subsequence. A person skilled in the art would appreciate that where the peptide can be a monomer, dimer, a trimer, etc. The use of the peptides of the present invention include use of peptides wherein the active fragment or fragments are complexed to one or more binding partners. Modified peptides which retain the activity of the peptides of the invention are encompassed within the scope of the present invention.

In another preferred embodiment, a TNFR25 peptide comprises at least one non-native amino acid residue or a non-amino acid molecule. A “non-native” amino acid residue comprises any change to an amino acid which is encoded by the TNFR25 nucleic acid sequence. Thus, a non-native amino acid residue or non-amino acid molecule comprises, without limitation: a chemical equivalent, analog, synthetic molecule, derivative, variant, substitution, peptide nucleic acid, a linker molecule, inorganic molecule etc.

The mutations can be introduced at the nucleic acid level or at the amino acid level. With respect to particular nucleic acid sequences, because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. If mutations at the nucleic acid level are introduced to encode a particular amino acid, then one or more nucleic acids are altered. For example proline is encoded by CCC, CCA, CCG, CCU; thus, one base change, e.g. CCC (proline) to GCC gives rise to alanine. Thus by way of example every natural or non-natural nucleic acid sequence herein which encodes a natural or non-natural polypeptide also describes every possible silent variation of the natural or non-natural nucleic acid. One of skill will recognize that each codon in a natural or non-natural nucleic acid (except AUG, which is ordinarily the only codon for methionine, and TGG, which is ordinarily the only codon for tryptophan) can be modified to yield a functionally identical molecule or a different molecule. Accordingly, each silent variation of a natural and non-natural nucleic acid which encodes a natural and non-natural polypeptide is implicit in each described sequence.

As to amino acid sequences, individual substitutions, deletions or additions to a nucleic acid, peptide, polypeptide, or protein sequence which alters, adds or deletes a single natural and non-natural amino acid or a small percentage of natural and non-natural amino acids in the encoded sequence, the alteration results in the deletion of an amino acid, addition of an amino acid, or substitution of a natural and non-natural amino acid with a chemically similar amino acid. Conservative substitution tables providing functionally similar natural amino acids are well known in the art. Such conservatively modified variants are in addition to and do not exclude polymorphic variants, interspecies homologs, and alleles of the methods and compositions described herein.

A “non-natural amino acid” refers to an amino acid that is not one of the 20 common amino acids or pyrolysine or selenocysteine. Other terms that may be used synonymously with the term “non-natural amino acid” is “non-naturally encoded amino acid,” “unnatural amino acid,” “non-naturally-occurring amino acid,” and variously hyphenated and non-hyphenated versions thereof. The term “non-natural amino acid” includes, but is not limited to, amino acids which occur naturally by modification of a naturally encoded amino acid (including but not limited to, the 20 common amino acids or pyrrolysine and selenocysteine) but are not themselves incorporated, without user manipulation, into a growing polypeptide chain by the translation complex. Examples of naturally-occurring amino acids that are not naturally-encoded include, but are not limited to, N-acetylglucosaminyl-L-serine, N-acetylglucosaminyl-L-threonine, and O-phosphotyrosine. Additionally, the term “non-natural amino acid” includes, but is not limited to, amino acids which do not occur naturally and may be obtained synthetically or may be obtained by modification of non-natural amino acids.

In some cases, the non-natural amino acid substitution(s) or incorporation(s) will be combined with other additions, substitutions, or deletions within the polypeptide to affect other chemical, physical, pharmacologic and/or biological traits. In some cases, the other additions, substitutions or deletions may increase the stability (including but not limited to, resistance to proteolytic degradation) of the polypeptide or increase affinity of the polypeptide for its appropriate receptor, ligand and/or binding proteins. In some cases, the other additions, substitutions or deletions may increase the solubility of the polypeptide. In some embodiments sites are selected for substitution with a naturally encoded or non-natural amino acid in addition to another site for incorporation of a non-natural amino acid for the purpose of increasing the polypeptide solubility following expression in recombinant host cells. In some embodiments, the polypeptides comprise another addition, substitution, or deletion that modulates affinity for the associated ligand, binding proteins, and/or receptor, modulates (including but not limited to, increases or decreases) receptor dimerization, stabilizes receptor dimers, modulates circulating half-life, modulates release or bio-availability, facilitates purification, or improves or alters a particular route of administration. Similarly, the non-natural amino acid polypeptide can comprise chemical or enzyme cleavage sequences, protease cleavage sequences, reactive groups, antibody-binding domains (including but not limited to, FLAG or poly-His) or other affinity based sequences (including but not limited to, FLAG, poly-His, GST, etc.) or linked molecules (including but not limited to, biotin) that improve detection (including but not limited to, GFP), purification, transport thru tissues or cell membranes, prodrug release or activation, size reduction, or other traits of the polypeptide.

The methods and compositions described herein include incorporation of one or more non-natural amino acids into a polypeptide. One or more non-natural amino acids may be incorporated at one or more particular positions which does not disrupt activity of the polypeptide. This can be achieved by making “conservative” substitutions, including but not limited to, substituting hydrophobic amino acids with non-natural or natural hydrophobic amino acids, bulky amino acids with non-natural or natural bulky amino acids, hydrophilic amino acids with non-natural or natural hydrophilic amino acids) and/or inserting the non-natural amino acid in a location that is not required for activity.

A variety of biochemical and structural approaches can be employed to select the desired sites for substitution with a non-natural amino acid within the polypeptide. Any position of the polypeptide chain is suitable for selection to incorporate a non-natural amino acid, and selection may be based on rational design or by random selection for any or no particular desired purpose. Selection of desired sites may be based on producing a non-natural amino acid polypeptide (which may be further modified or remain unmodified) having any desired property or activity, including but not limited to agonists, super-agonists, partial agonists, inverse agonists, antagonists, receptor binding modulators, receptor activity modulators, modulators of binding to binder partners, binding partner activity modulators, binding partner conformation modulators, dimer or multimer formation, no change to activity or property compared to the native molecule, or manipulating any physical or chemical property of the polypeptide such as solubility, aggregation, or stability. For example, locations in the polypeptide required for biological activity of a polypeptide can be identified using methods including, but not limited to, point mutation analysis, alanine scanning or homolog scanning methods. Residues other than those identified as critical to biological activity by methods including, but not limited to, alanine or homolog scanning mutagenesis may be good candidates for substitution with a non-natural amino acid depending on the desired activity sought for the polypeptide. Alternatively, the sites identified as critical to biological activity may also be good candidates for substitution with a non-natural amino acid, again depending on the desired activity sought for the polypeptide. Another alternative would be to make serial substitutions in each position on the polypeptide chain with a non-natural amino acid and observe the effect on the activities of the polypeptide. Any means, technique, or method for selecting a position for substitution with a non-natural amino acid into any polypeptide is suitable for use in the methods, techniques and compositions described herein.

The structure and activity of naturally-occurring mutants of a polypeptide that contain deletions can also be examined to determine regions of the protein that are likely to be tolerant of substitution with a non-natural amino acid. Once residues that are likely to be intolerant to substitution with non-natural amino acids have been eliminated, the impact of proposed substitutions at each of the remaining positions can be examined using methods including, but not limited to, the three-dimensional structure of the relevant polypeptide, and any associated ligands or binding proteins. X-ray crystallographic and NMR structures of many polypeptides are available in the Protein Data Bank (PDB, www.rcsb.org), a centralized database containing three-dimensional structural data of large molecules of proteins and nucleic acids, one can be used to identify amino acid positions that can be substituted with non-natural amino acids. In addition, models may be made investigating the secondary and tertiary structure of polypeptides, if three-dimensional structural data is not available. Thus, the identity of amino acid positions that can be substituted with non-natural amino acids can be readily obtained. Exemplary sites of incorporation of a non-natural amino acid include, but are not limited to, those that are excluded from potential receptor binding regions, or regions for binding to binding proteins or ligands may be fully or partially solvent exposed, have minimal or no hydrogen-bonding interactions with nearby residues, may be minimally exposed to nearby reactive residues, and/or may be in regions that are highly flexible as predicted by the three-dimensional crystal structure of a particular polypeptide with its associated receptor, ligand or binding proteins.

A wide variety of non-natural amino acids can be substituted for, or incorporated into, a given position in a polypeptide. By way of example, a particular non-natural amino acid may be selected for incorporation based on an examination of the three dimensional crystal structure of a polypeptide with its associated ligand, receptor and/or binding proteins, a preference for conservative substitutions

As further used herein, a “chemical equivalent” of a peptide of the invention is a molecule which possesses the same desired activity, e.g. immunological activity, as peptides described herein, and exhibits a trivial chemical different, or a molecule which is converted, under mild conditions, into a peptide of the invention (e.g., esters, ethers, reduction products, and complexes of the peptides of the invention).

Additionally, as used herein, “conservative substitutions” are those amino acid substitutions which are functionally equivalent to the substituted amino acid residue, either because they have similar polarity or steric arrangement, or because they belong to the same class as the substituted residue (e.g., hydrophobic, acidic, or basic). The term “conservative substitutions”, as defined herein, includes substitutions having an inconsequential effect on the ability of the peptide of the invention to enhance innate immunity. Examples of conservative substitutions include the substitution of a polar (hydrophilic) residue for another arginine/lysine, glutamine/asparagine, or threonine/serine); the substitution of a non-polar (hydrophobic) residue (e.g. isoleucine, leucine, methionine, phenylalanine, tyrosine) for another, the substitution of an acidic residue (e.g., aspartic acid or glutamic acid) for another; or the substitution of a basic residue (e.g., arginine, histidine, lysine or ornithine) for another.

The term “analogue”, as used herein, includes any peptide having an amino acid sequence substantially identical to a sequence described herein, in which at least one residue has been conservatively substituted with a functionally-similar residue. An “analogue” includes functional variants and obvious chemical equivalents of an amino acid sequence of a TNFR25 peptide. As further used herein, the term “functional variant” refers to the activity of a peptide that demonstrates an ability to modulate immunity. An “analogue” further includes any pharmaceutically-acceptable salt of an analogue as described herein.

A “derivative”, also refers to a peptide of the invention having one or more amino acids chemically derivatized by reaction of a functional side group. Exemplary derivatized molecules include, without limitation, peptide molecules in which free amino groups have been derivatized to form salts or amides, by adding acetyl groups, amine hydrochlorides, carbobenzoxy groups, chloroacetyl groups, formyl groups, p-toluene sulfonyl groups, or t-butyloxycarbonyl groups. Free hydroxyl groups may be derivatized to form O-acyl or O-alkyl derivatives. Furthermore, free carboxyl groups may be derivatized to form salts, esters (e.g., methyl and ethyl esters), or hydrazides. Thus, a “derivative” further includes any pharmaceutically-acceptable salt of a derivative as described herein.

In one embodiment of the present invention, the isolated peptide of the invention has a modified C-terminus and/or a modified N-terminus For example, the isolated peptide may have an amidated C-terminus For example, the amino terminus can be acetylated (Ac) or the carboxy terminus can be amidated (NH₂). However, in one embodiment of the invention, the peptides of the invention are preferably not acetylated if such a modification would result in loss of desired immunological activity Amino terminus modifications include methylating (i.e., —NHCH₃ or —NH(CH₃), acetylating, adding a carbobenzoyl group, or blocking the amino terminus with any blocking group containing a carboxylase functionality defined by RCOO—, where R is selected from the group consisting of naphthyl, acridinyl, steroidyl, and similar groups. Carboxy terminus modifications include replacing the free acid with a carboxamide group or forming a cyclic lactam at the carboxy terminus to introduce structural constraints.

In one embodiment backbone substitutions can be made, such as NH to NCH₃. The isolated peptide may also be a modification (e.g., a point mutation, such as an insertion or a deletion, or a truncation). By way of example, the peptide may comprise an amino acid sequence comprising a modified residue by at least one point insertion of a D amino acid as long as desired TNFR25 activity is retained. In particular, proline analogs in which the ring size of the proline residue is changed from 5 members to 4, 6, or 7 members can be employed. Cyclic groups can be saturated or unsaturated, and if unsaturated, can be aromatic or non-aromatic.

In another preferred embodiment, the naturally occurring side chains of the 20 genetically encoded amino acids (or D amino acids) are replaced with other side chains with similar properties, for instance with groups such as alkyl, lower alkyl, cyclic 4-, 5-, 6-, to 7-membered alkyl amide, amide lower alkyl amide di(lower alkyl), lower alkoxy, hydroxy, carboxy and the lower ester derivatives thereof, and with 4-, 5-, 6-, to 7-membered heterocyclic.

Such substitutions can include but are not necessarily limited to: (1) non-standard positively charged amino acids, like: ornithine, Nlys; N-(4-aminobutyl)-glycine which has the lysine side chain attached to the “N-terminus” and compounds with aminopropyl or aminoethyl groups attached to the amino group of glycine. (2), Non-naturally occurring amino acids with no net charge and side-chains similar to arginine, such as, Cit; citrulline and Hci; citrulline with one more methylene group; (3) non-standard non-naturally occurring amino acids with OH (e.g., like serine), such as, hSer; homoserine (one more methylen group, Hyp; hydroxyproline, Val(βOH); hydroxyvaline, Pen; penicillamin, (Val(βSH); (4) proline derivatives, such as, D-Pro, such as, 3,4-dehydroproline, Pyr; pyroglutamine (proline with C═O in ring), Proline with fluorine substitutions on the ring, 1,3-thiazolidine-+carboxylic acid (proline with S in ring); (5) Histidine derivative, such as, Thi; beta-(2-thienyl)-alanine; or (6) alkyl derivatives, such as, Abu; 2-aminobutyric acid (ethyl group on Ca), Nva; norvaline (propyl group on Cα), Nle; norleucine (butyl group on Cα), Hol; homoleucine (propyl group on Cα), Aib, alpha-aminoisobutyric acid (valine without methylene group). A person skilled in the art would appreciate that those substitutions that retain the activity of the parent peptide/sequence.

In another alternative embodiment, the C-terminal carboxyl group or a C-terminal ester can be induced to cyclize by internal displacement of the —OH or the ester (—OR) of the carboxyl group or ester respectively with the N-terminal amino group to form a cyclic peptide. For example, after synthesis and cleavage to give the peptide acid, the free acid is converted to an activated ester by an appropriate carboxyl group activator such as dicyclohexylcarbodiimide (DCC) in solution, for example, in methylene chloride (CH₂Cl₂), dimethyl formamide (DMF) mixtures. The cyclic peptide is then formed by internal displacement of the activated ester with the N-terminal amine. Internal cyclization as opposed to polymerization can be enhanced by use of very dilute solutions. Such methods are well known in the art.

The peptides of the invention can be cyclized, or a desamino or descarboxy residue at the termini of the peptide can be incorporated, so that there is no terminal amino or carboxyl group, to decrease susceptibility to proteases or to restrict the conformation of the peptide. C-terminal functional groups of the compounds of the present invention include amide, amide lower alkyl, amide di(lower alkyl), lower alkoxy, hydroxy, and carboxy, and the lower ester derivatives thereof, and the pharmaceutically acceptable salts thereof.

The peptides of the invention can be cyclized by adding an N and/or C terminal cysteine and cyclizing the peptide through disulfide linkages or other side chain interactions.

A desamino or descarboxy residue at the termini of the peptide can be incorporated, so that there is no terminal amino or carboxyl group, to decrease susceptibility to proteases or to restrict the conformation of the peptide.

Antisense TNFR25 Oligonucleotides: In another preferred embodiment, the functions and/or expression of TNFR25 in a cell or patient are modulated by targeting TNFR25 molecules.

In a preferred embodiment, an oligonucleotide comprises at least five consecutive bases complementary to a TNFR25 nucleic acid sequence, wherein the oligonucleotide specifically hybridizes to and modulates expression and/or function of TNFR25 in vivo or in vitro. In another preferred embodiment, the oligomeric compounds of the present invention also include variants in which a different base is present at one or more of the nucleotide positions in the compound. For example, if the first nucleotide is an adenosine, variants may be produced which contain thymidine, guanosine or cytidine at this position. This may be done at any of the positions of the oligonucleotide. These compounds are then tested using the methods described herein to determine their ability to inhibit expression of a target nucleic acid.

In some embodiments, homology, sequence identity or complementarity, between the oligonucleotide and target is from about 50% to about 60%. In some embodiments, homology, sequence identity or complementarity, is from about 60% to about 70%. In some embodiments, homology, sequence identity or complementarity, is from about 70% to about 80%. In some embodiments, homology, sequence identity or complementarity, is from about 80% to about 90%. In some embodiments, homology, sequence identity or complementarity, is about 90%, about 92%, about 94%, about 95%, about 96%, about 97%, about 98%, about 99% or about 100%.

In another preferred embodiment, an oligonucleotide comprises combinations of phosphorothioate internucleotide linkages and at least one internucleotide linkage selected from the group consisting of: alkylphosphonate, phosphorodithioate, alkylphosphonothioate, phosphoramidate, carbamate, carbonate, phosphate triester, acetamidate, carboxymethyl ester, and/or combinations thereof.

In another preferred embodiment, an oligonucleotide optionally comprises at least one modified nucleobase comprising, peptide nucleic acids, locked nucleic acid (LNA) molecules, analogues, derivatives and/or combinations thereof.

An oligonucleotide is specifically hybridizable when binding of the compound to the target nucleic acid interferes with the normal function of the target nucleic acid to cause a loss of activity, and there is a sufficient degree of complementarity to avoid non-specific binding of the oligonucleotide to non-target nucleic acid sequences under conditions in which specific binding is desired. Such conditions include, i.e., physiological conditions in the case of in vivo assays or therapeutic treatment, and conditions in which assays are performed in the case of in vitro assays.

An oligonucleotide, whether DNA, RNA, chimeric, substituted etc, is specifically hybridizable when binding of the compound to the target DNA or RNA molecule interferes with the normal function of the target DNA or RNA to cause a loss of utility, and there is a sufficient degree of complementarily to avoid non-specific binding of the oligonucleotide to non-target sequences under conditions in which specific binding is desired, i.e., under physiological conditions in the case of in vivo assays or therapeutic treatment, and in the case of in vitro assays, under conditions in which the assays are performed.

The specificity and sensitivity of antisense is also harnessed by those of skill in the art for therapeutic uses. Antisense oligonucleotides have been employed as therapeutic moieties in the treatment of disease states in animals and man. Antisense oligonucleotides have been safely and effectively administered to humans and numerous clinical trials are presently underway. It is thus established that oligonucleotides can be useful therapeutic modalities that can be configured to be useful in treatment regimes for treatment of cells, tissues and animals, especially humans.

In embodiments of the present invention oligomeric oligonucleotides, particularly oligonucleotides, bind to target nucleic acid molecules and modulate the expression and/or function of molecules encoded by a target gene. The functions of DNA to be interfered comprise, for example, replication and transcription. The functions of RNA to be interfered comprise all vital functions such as, for example, translocation of the RNA to the site of protein translation, translation of protein from the RNA, splicing of the RNA to yield one or more mRNA species, and catalytic activity which may be engaged in or facilitated by the RNA. The functions may be up-regulated or inhibited depending on the functions desired.

The oligonucleotides, include, antisense oligomeric compounds, antisense oligonucleotides, external guide sequence (EGS) oligonucleotides, alternate splicers, primers, probes, and other oligomeric compounds that hybridize to at least a portion of the target nucleic acid. As such, these compounds may be introduced in the form of single-stranded, double-stranded, partially single-stranded, or circular oligomeric compounds.

Targeting an oligonucleotide to a particular nucleic acid molecule, in the context of this invention, can be a multistep process. The process usually begins with the identification of a target nucleic acid whose function is to be modulated. This target nucleic acid may be, for example, a cellular gene (or mRNA transcribed from the gene) whose expression is associated with a particular disorder or disease state, or a nucleic acid molecule from an infectious agent.

The targeting process usually also includes determination of at least one target region, segment, or site within the target nucleic acid for the antisense interaction to occur such that the desired effect, e.g., modulation of expression, will result. Within the context of the present invention, the term “region” is defined as a portion of the target nucleic acid having at least one identifiable structure, function, or characteristic. Within regions of target nucleic acids are segments. “Segments” are defined as smaller or sub-portions of regions within a target nucleic acid. “Sites,” as used in the present invention, are defined as positions within a target nucleic acid.

Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes has a translation initiation codon having the RNA sequence 5′-GUG, 5′-UUG or 5′-CUG; and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). Eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA transcribed from a gene encoding TNFR25, regardless of the sequence(s) of such codons. A translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively).

The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon. Consequently, the “start codon region” (or “translation initiation codon region”) and the “stop codon region” (or “translation termination codon region”) are all regions that may be targeted effectively with the antisense compounds of the present invention.

The open reading frame (ORF) or “coding region,” which is known in the art to refer to the region between the translation initiation codon and the translation termination codon, is also a region which may be targeted effectively. Within the context of the present invention, a targeted region is the intragenic region encompassing the translation initiation or termination codon of the open reading frame (ORF) of a gene.

Another target region includes the 5′ untranslated region (5′UTR), known in the art to refer to the portion of an mRNA in the 5′ direction from the translation initiation codon, and thus including nucleotides between the 5′ cap site and the translation initiation codon of an mRNA (or corresponding nucleotides on the gene). Still another target region includes the 3′ untranslated region (3′UTR), known in the art to refer to the portion of an mRNA in the 3′ direction from the translation termination codon, and thus including nucleotides between the translation termination codon and 3′ end of an mRNA (or corresponding nucleotides on the gene). The 5′ cap site of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap site. Another target region for this invention is the 5′ cap region.

Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence. In one embodiment, targeting splice sites, i.e., intron-exon junctions or exon-intron junctions, is particularly useful in situations where aberrant splicing is implicated in disease, or where an overproduction of a particular splice product is implicated in disease. An aberrant fusion junction due to rearrangement or deletion is another embodiment of a target site. mRNA transcripts produced via the process of splicing of two (or more) mRNAs from different gene sources are known as “fusion transcripts”. Introns can be effectively targeted using antisense compounds targeted to, for example, DNA or pre-mRNA.

In another preferred embodiment, the antisense oligonucleotides bind to coding and/or non-coding regions of a target polynucleotide and modulate the expression and/or function of the target molecule.

In another preferred embodiment, the antisense oligonucleotides bind to natural antisense polynucleotides and modulate the expression and/or function of the target molecule.

In another preferred embodiment, the antisense oligonucleotides bind to sense polynucleotides and modulate the expression and/or function of the target molecule.

Alternative RNA transcripts can be produced from the same genomic region of DNA. These alternative transcripts or “pre-mRNA variants” are transcripts produced from the same genomic DNA that differ from other transcripts produced from the same genomic DNA in either their start or stop position and contain both intronic and exonic sequence.

Upon excision of one or more exon or intron regions, or portions thereof during splicing, pre-mRNA variants produce smaller “mRNA variants”. Consequently, mRNA variants are processed pre-mRNA variants and each unique pre-mRNA variant must always produce a unique mRNA variant as a result of splicing. These mRNA variants are also known as “alternative splice variants”. If no splicing of the pre-mRNA variant occurs then the pre-mRNA variant is identical to the mRNA variant.

Variants can be produced through the use of alternative signals to start or stop transcription. Pre-mRNAs and mRNAs can possess more that one start codon or stop codon. Variants that originate from a pre-mRNA or mRNA that use alternative start codons are known as “alternative start variants” of that pre-mRNA or mRNA. Those transcripts that use an alternative stop codon are known as “alternative stop variants” of that pre-mRNA or mRNA. One specific type of alternative stop variant is the “polyA variant” in which the multiple transcripts produced result from the alternative selection of one of the “polyA stop signals” by the transcription machinery, thereby producing transcripts that terminate at unique polyA sites. Within the context of the invention, the types of variants described herein are also embodiments of target nucleic acids.

The locations on the target nucleic acid to which the antisense compounds hybridize are defined as at least a 5-nucleobase portion of a target region to which an active antisense compound is targeted.

While the specific sequences of certain exemplary target segments are set forth herein, one of skill in the art will recognize that these serve to illustrate and describe particular embodiments within the scope of the present invention. Additional target segments are readily identifiable by one having ordinary skill in the art in view of this disclosure.

Target segments 5-100 nucleobases in length comprising a stretch of at least five (5) consecutive nucleobases selected from within the illustrative preferred target segments are considered to be suitable for targeting as well.

Target segments can include DNA or RNA sequences that comprise at least the 5 consecutive nucleobases from the 5′-terminus of one of the illustrative preferred target segments (the remaining nucleobases being a consecutive stretch of the same DNA or RNA beginning immediately upstream of the 5′-terminus of the target segment and continuing until the DNA or RNA contains about 5 to about 100 nucleobases). Similarly preferred target segments are represented by DNA or RNA sequences that comprise at least the 5 consecutive nucleobases from the 3′-terminus of one of the illustrative preferred target segments (the remaining nucleobases being a consecutive stretch of the same DNA or RNA beginning immediately downstream of the 3′-terminus of the target segment and continuing until the DNA or RNA contains about 5 to about 100 nucleobases). One having skill in the art armed with the target segments illustrated herein will be able, without undue experimentation, to identify further preferred target segments.

Once one or more target regions, segments or sites have been identified, antisense compounds are chosen which are sufficiently complementary to the target, i.e., hybridize sufficiently well and with sufficient specificity, to give the desired effect.

In embodiments of the invention the oligonucleotides bind to an antisense strand of a particular target. The oligonucleotides are at least 5 nucleotides in length and can be synthesized so each oligonucleotide targets overlapping sequences such that oligonucleotides are synthesized to cover the entire length of the target polynucleotide. The targets also include coding as well as non coding regions.

According to the present invention, antisense compounds include antisense oligonucleotides, ribozymes, external guide sequence (EGS) oligonucleotides, siRNA compounds, single- or double-stranded RNA interference (RNAi) compounds such as siRNA compounds, and other oligomeric compounds which hybridize to at least a portion of the target nucleic acid and modulate its function. As such, they may be DNA, RNA, DNA-like, RNA-like, or mixtures thereof, or may be mimetics of one or more of these. These compounds may be single-stranded, double-stranded, circular or hairpin oligomeric compounds and may contain structural elements such as internal or terminal bulges, mismatches or loops. Antisense compounds are routinely prepared linearly but can be joined or otherwise prepared to be circular and/or branched. Antisense compounds can include constructs such as, for example, two strands hybridized to form a wholly or partially double-stranded compound or a single strand with sufficient self-complementarity to allow for hybridization and formation of a fully or partially double-stranded compound. The two strands can be linked internally leaving free 3′ or 5′ termini or can be linked to form a continuous hairpin structure or loop. The hairpin structure may contain an overhang on either the 5′ or 3′ terminus producing an extension of single stranded character. The double stranded compounds optionally can include overhangs on the ends. Further modifications can include conjugate groups attached to one of the termini, selected nucleobase positions, sugar positions or to one of the intemucleoside linkages. Alternatively, the two strands can be linked via a non-nucleic acid moiety or linker group. When formed from only one strand, dsRNA can take the form of a self-complementary hairpin-type molecule that doubles back on itself to form a duplex. Thus, the dsRNAs can be fully or partially double stranded. Specific modulation of gene expression can be achieved by stable expression of dsRNA hairpins in transgenic cell lines, however, in some embodiments, the gene expression or function is up regulated. When formed from two strands, or a single strand that takes the form of a self-complementary hairpin-type molecule doubled back on itself to form a duplex, the two strands (or duplex-forming regions of a single strand) are complementary RNA strands that base pair in Watson-Crick fashion.

Once introduced to a system, the compounds of the invention may elicit the action of one or more enzymes or structural proteins to effect cleavage or other modification of the target nucleic acid or may work via occupancy-based mechanisms. In general, nucleic acids (including oligonucleotides) may be described as “DNA-like” (i.e., generally having one or more 2′-deoxy sugars and, generally, T rather than U bases) or “RNA-like” (i.e., generally having one or more 2′-hydroxyl or 2′-modified sugars and, generally U rather than T bases). Nucleic acid helices can adopt more than one type of structure, most commonly the A- and B-forms. It is believed that, in general, oligonucleotides which have B-form-like structure are “DNA-like” and those which have A-form-like structure are “RNA-like.” In some (chimeric) embodiments, an antisense compound may contain both A- and B-form regions.

In another preferred embodiment, the desired oligonucleotides or antisense compounds, comprise at least one of: antisense RNA, antisense DNA, chimeric antisense oligonucleotides, antisense oligonucleotides comprising modified linkages, interference RNA (RNAi), short interfering RNA (siRNA); a micro, interfering RNA (miRNA); a small, temporal RNA (stRNA); or a short, hairpin RNA (shRNA); small RNA-induced gene activation (RNAa); small activating RNAs (saRNAs), or combinations thereof.

dsRNA can also activate gene expression, a mechanism that has been termed “small RNA-induced gene activation” or RNAa. dsRNAs targeting gene promoters induce potent transcriptional activation of associated genes. RNAa was demonstrated in human cells using synthetic dsRNAs, termed “small activating RNAs” (saRNAs). It is currently not known whether RNAa is conserved in other organisms.

Small double-stranded RNA (dsRNA), such as small interfering RNA (siRNA) and microRNA (miRNA), have been found to be the trigger of an evolutionary conserved mechanism known as RNA interference (RNAi). RNAi invariably leads to gene silencing via remodeling chromatin to thereby suppress transcription, degrading complementary mRNA, or blocking protein translation. dsRNAs may also act as small activating RNAs (saRNA). Without wishing to be bound by theory, by targeting sequences in gene promoters, saRNAs would induce target gene expression in a phenomenon referred to as dsRNA-induced transcriptional activation (RNAa).

RNA interference (RNAi) has become a powerful tool for blocking gene expression in mammals and mammalian cells. This approach requires the delivery of small interfering RNA (siRNA) either as RNA itself or as DNA, using an expression plasmid or virus and the coding sequence for small hairpin RNAs that are processed to siRNAs. This system enables efficient transport of the pre-siRNAs to the cytoplasm where they are active and permit the use of regulated and tissue specific promoters for gene expression.

In a preferred embodiment, an oligonucleotide or antisense compound comprises an oligomer or polymer of ribonucleic acid (RNA) and/or deoxyribonucleic acid (DNA), or a mimetic, chimera, analog or homolog thereof. This term includes oligonucleotides composed of naturally occurring nucleobases, sugars and covalent internucleoside (backbone) linkages as well as oligonucleotides having non-naturally occurring portions which function similarly. Such modified or substituted oligonucleotides are often desired over native forms because of desirable properties such as, for example, enhanced cellular uptake, enhanced affinity for a target nucleic acid and increased stability in the presence of nucleases.

According to the present invention, the oligonucleotides or “antisense compounds” include antisense oligonucleotides (e.g. RNA, DNA, mimetic, chimera, analog or homolog thereof), ribozymes, external guide sequence (EGS) oligonucleotides, siRNA compounds, single- or double-stranded RNA interference (RNAi) compounds such as siRNA compounds, saRNA, aRNA, and other oligomeric compounds which hybridize to at least a portion of the target nucleic acid and modulate its function. As such, they may be DNA, RNA, DNA-like, RNA-like, or mixtures thereof, or may be mimetics of one or more of these. These compounds may be single-stranded, double-stranded, circular or hairpin oligomeric compounds and may contain structural elements such as internal or terminal bulges, mismatches or loops. Antisense compounds are routinely prepared linearly but can be joined or otherwise prepared to be circular and/or branched. Antisense compounds can include constructs such as, for example, two strands hybridized to form a wholly or partially double-stranded compound or a single strand with sufficient self-complementarity to allow for hybridization and formation of a fully or partially double-stranded compound. The two strands can be linked internally leaving free 3′ or 5′ termini or can be linked to form a continuous hairpin structure or loop. The hairpin structure may contain an overhang on either the 5′ or 3′ terminus producing an extension of single stranded character. The double stranded compounds optionally can include overhangs on the ends. Further modifications can include conjugate groups attached to one of the termini, selected nucleobase positions, sugar positions or to one of the internucleoside linkages. Alternatively, the two strands can be linked via a non-nucleic acid moiety or linker group. When formed from only one strand, dsRNA can take the form of a self-complementary hairpin-type molecule that doubles back on itself to form a duplex. Thus, the dsRNAs can be fully or partially double stranded. Specific modulation of gene expression can be achieved by stable expression of dsRNA hairpins in transgenic cell lines (Hammond et al., Nat. Rev. Genet., 1991, 2, 110-119; Matzke et al., Curr. Opin. Genet. Dev., 2001, 11, 221-227; Sharp, Genes Dev., 2001, 15, 485-490). When formed from two strands, or a single strand that takes the form of a self-complementary hairpin-type molecule doubled back on itself to form a duplex, the two strands (or duplex-forming regions of a single strand) are complementary RNA strands that base pair in Watson-Crick fashion.

The antisense compounds in accordance with this invention can comprise an antisense portion from about 5 to about 80 nucleobases (i.e. from about 5 to about 80 linked nucleosides) in length. This refers to the length of the antisense strand or portion of the antisense compound. In other words, a single-stranded antisense compound of the invention comprises from 5 to about 80 nucleobases, and a double-stranded antisense compound of the invention (such as a dsRNA, for example) comprises an antisense strand or portion of 5 to about 80 nucleobases in length. One of ordinary skill in the art will appreciate that this comprehends antisense portions of 5, 6, 7,8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleobases in length, or any range therewithin.

In one embodiment, the antisense compounds of the invention have antisense portions of 10 to 50 nucleobases in length. One having ordinary skill in the art will appreciate that this embodies oligonucleotides having antisense portions of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleobases in length, or any range therewithin. In some embodiments, the oligonucleotides are 15 nucleobases in length.

In one embodiment, the antisense or oligonucleotide compounds of the invention have antisense portions of 12 or 13 to 30 nucleobases in length. One having ordinary skill in the art will appreciate that this embodies antisense compounds having antisense portions of 12, 13, 14, 15, 16, 17, 18, 19, 20, 21, 22, 23, 24, 25, 26, 27, 28, 29 or 30 nucleobases in length, or any range therewithin.

Certain preferred oligonucleotides of this invention are chimeric oligonucleotides. “Chimeric oligonucleotides” or “chimeras,” in the context of this invention, are oligonucleotides which contain two or more chemically distinct regions, each made up of at least one nucleotide. These oligonucleotides typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of antisense modulation of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art. In one preferred embodiment, a chimeric oligonucleotide comprises at least one region modified to increase target binding affinity, and, usually, a region that acts as a substrate for RNAse H. Affinity of an oligonucleotide for its target is routinely determined by measuring the Tₘ of an oligonucleotide/target pair, which is the temperature at which the oligonucleotide and target dissociate; dissociation is detected spectrophotometrically. The higher the Tₘ, the greater the affinity of the oligonucleotide for the target.

Chimeric antisense compounds of the invention may be formed as composite structures of two or more oligonucleotides, modified oligonucleotides, oligonucleosides and/or oligonucleotide mimetics as described above. Such; compounds have also been referred to in the art as hybrids or gapmers. Representative United States patents that teach the preparation of such hybrid structures comprise, but are not limited to, U.S. Pat. Nos. 5,013,830; 5,149,797; 5, 220,007; 5,256,775; 5,366,878; 5,403,711; 5,491,133; 5,565,350; 5,623,065; 5,652,355; 5,652,356; and 5,700,922, each of which is herein incorporated by reference.

In another preferred embodiment, the region of the oligonucleotide which is modified comprises at least one nucleotide modified at the 2′ position of the sugar, most preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tₘ (i.e., higher target binding affinity) than; 2′-deoxyoligonucleotides against a given target. The effect of such increased affinity is to greatly enhance RNAi oligonucleotide inhibition of gene expression. RNAse H is a cellular endonuclease that cleaves the RNA strand of RNA:DNA duplexes; activation of this enzyme therefore results in cleavage of the RNA target, and thus can greatly enhance the efficiency of RNAi inhibition. Cleavage of the RNA target can be routinely demonstrated by gel electrophoresis. In another preferred embodiment, the chimeric oligonucleotide is also modified to enhance nuclease resistance. Cells contain a variety of exo- and endo-nucleases which can degrade nucleic acids. A number of nucleotide and nucleoside modifications have been shown to make the oligonucleotide into which they are incorporated more resistant to nuclease digestion than the native oligodeoxynucleotide. Nuclease resistance is routinely measured by incubating oligonucleotides with cellular extracts or isolated nuclease solutions and measuring the extent of intact oligonucleotide remaining over time, usually by gel electrophoresis. Oligonucleotides which have been modified to enhance their nuclease resistance survive intact for a longer time than unmodified oligonucleotides. A variety of oligonucleotide modifications have been demonstrated to enhance or confer nuclease resistance. Oligonucleotides which contain at least one phosphorothioate modification are presently more preferred. In some cases, oligonucleotide modifications which enhance target binding affinity are also, independently, able to enhance nuclease resistance. Some desirable modifications can be found in De Mesmaeker et al. Acc. Chem. Res. 1995, 28:366-374.

Specific examples of some preferred oligonucleotides envisioned for this invention include those comprising modified backbones, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones, particularly CH₂—NH—O—CH₂, CH,—N(CH₃)—O—CH₂ [known as a methylene(methylimino) or MMI backbone], CH₂—O—N(CH₃)—CH₂, CH₂—N(CH₃)—N(CH₃)—CH₂ and O—N (CH₃)—CH₂—CH, backbones, wherein the native phosphodiester backbone is represented as O—P—O—CH,). The amide backbones disclosed by De Mesmaeker et al. Acc. Chem. Res. 1995, 28:366-374) are also preferred. Also preferred are oligonucleotides having morpholino backbone structures (Summerton and Weller, U.S. Pat. No. 5,034,506). In other preferred embodiments, such as the peptide nucleic acid (PNA) backbone, the phosphodiester backbone of the oligonucleotide is replaced with a polyamide backbone, the nucleobases being bound directly or indirectly to the aza nitrogen atoms of the polyamide backbone (Nielsen et al. Science 1991, 254, 1497). Oligonucleotides may also comprise one or more substituted sugar moieties. Preferred oligonucleotides comprise one of the following at the 2′ position: OH, SH, SCH₃, F, OCN, OCH₃OCH₃, OCH₃O(CH₂)ₙCH₃, O(CH₂)ₙNH₂ or O(CH₂)ₙCH₃ where n is from 1 to about 10; C₁ to C₁₀ lower alkyl, alkoxyalkoxy, substitute lower alkyl, alkaryl or aralkyl; Cl; Br; CN; CF₃; OCF₃; O-, S-, or N-alkyl; O-, S-, or N-alkenyl; SOCH₃; SO₂CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes T-methoxyethoxy [2′-O—CH₂CH₂OCH₃, also known as 2′-O-(2-methoxyethyl)] (Martin et al., Helv. Chim. Acta, 1995, 78, 486). Other preferred modifications include 2′-methoxy (2′-O—CH₃), 2′-propoxy (2′-OCH₂CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

Oligonucleotides may also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N₆ (6-aminohexyl)adenine and 2,6-diaminopurine. Kornberg, A., DNA Replication, W. H. Freeman & Co., San Francisco, 1980, pp 75-77; Gebeyehu, G., et al. Nucl. Acids Res. 1987, 15:4513). A “universal” base known in the art, e.g., inosine, may be included. 5-Me-C substitutions have been shown to increase nucleic acid duplex stability by 0.6-1.2° C. (Sanghvi, Y. S., in Crooke, S. T. and Lebleu, B., eds., Antisense Research and Applications, CRC Press, Boca Raton, 1993, pp. 276-278) and are presently preferred base substitutions.

Another modification of the oligonucleotides of the invention involves chemically linking to the oligonucleotide one or more moieties or conjugates which enhance the activity or cellular uptake of the oligonucleotide. Such moieties include but are not limited to lipid moieties such as a cholesterol moiety, a cholesteryl moiety (Letsinger et al., Proc. Natl. Acad. Sci. USA 1989, 86, 6553), cholic acid (Manoharan et al. Bioorg. Med. Chem. Let. 1994, 4, 1053), a thioether, e.g., hexyl-S-tritylthiol (Manoharan et al. Ann. N.Y. Acad. Sci. 1992, 660, 306; Manoharan et al. Bioorg. Med. Chem. Let. 1993, 3, 2765), a thiocholesterol (Oberhauser et al., Nucl. Acids Res. 1992, 20, 533), an aliphatic chain, e.g., dodecandiol or undecyl residues (Saison-Behmoaras et al. EMBO J. 1991, 10, 111; Kabanov et al. FEBS Lett. 1990, 259, 327; Svinarchuk et al. Biochimie 1993, 75, 49), a phospholipid, e.g., di-hexadecyl-rac-glycerol or triethylammonium 1,2-di-O-hexadecyl-rac-glycero-3-H-phosphonate (Manoharan et al. Tetrahedron Lett. 1995, 36, 3651; Shea et al. Nucl. Acids Res. 1990, 18, 3777), a polyamine or a polyethylene glycol chain (Manoharan et al. Nucleosides & Nucleotides 1995, 14, 969), or adamantane acetic acid (Manoharan et al. Tetrahedron Lett. 1995, 36, 3651). Oligonucleotides comprising lipophilic moieties, and methods for preparing such oligonucleotides are known in the art, for example, U.S. Pat. Nos. 5,138,045, 5,218,105 and 5,459,255.

It is not necessary for all positions in a given oligonucleotide to be uniformly modified, and in fact more than one of the aforementioned modifications may be incorporated in a single oligonucleotide or even at within a single nucleoside within an oligonucleotide. The present invention also includes oligonucleotides which are chimeric oligonucleotides as hereinbefore defined.

In another embodiment, the nucleic acid molecule of the present invention is conjugated with another moiety including but not limited to abasic nucleotides, polyether, polyamine, polyamides, peptides, carbohydrates, lipid, or polyhydrocarbon compounds. Those skilled in the art will recognize that these molecules can be linked to one or more of any nucleotides comprising the nucleic acid molecule at several positions on the sugar, base or phosphate group.

In accordance with the invention, use of modifications such as the use of LNA monomers to enhance the potency, specificity and duration of action and broaden the routes of administration of oligonucleotides comprised of current chemistries such as MOE, ANA, FANA, PS etc (ref: Recent advances in the medical chemistry of antisense oligonucleotide by Uhlman, Current Opinions in Drug Discovery & Development 2000 Vol 3 No 2). This can be achieved by substituting some of the monomers in the current oligonucleotides by LNA monomers. The LNA modified oligonucleotide may have a size similar to the parent compound or may be larger or preferably smaller. It is preferred that such LNA-modified oligonucleotides contain less than about 70%, more preferably less than about 60%, most preferably less than about 50% LNA monomers and that their sizes are between about 5 and 25 nucleotides, more preferably between about 12 and 20 nucleotides.

Preferred modified oligonucleotide backbones comprise, but not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH, component parts.

In other preferred oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone.

In another preferred embodiment of the invention the oligonucleotides with phosphorothioate backbones and oligonucleotides with heteroatom backbones, and in particular —CH2—NH—O—CH₂—, —CH₂—N(CH₃)—O-CH₂— known as a methylene(methylimino) or MMI backbone, —CH₂—O—N(CH₃)—CH₂—, —CH₂N(CH₃)—N(CH₃)CH₂— and —O—N(CH₃)—CH₂—CH₂— wherein the native phosphodiester backbone is represented as —O—P—O—CH₂— of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. Preferred oligonucleotides comprise one of the following at the T position: OH; F; O, S-, or N-alkyl; O-, S-, or N-alkenyl; O, S-or N-alkynyl; or O alkyl-O-alkyl, wherein the alkyl, alkenyl and alkynyl may be substituted or unsubstituted C to CO alkyl or C, to CO alkenyl and alkynyl. Particularly preferred are O (CH₂)ₙOₘCH₃, O(CH₂)ₙ, OCH₃, O(CH₂)ₙNH₂, O(CH₂)nCH₃, O(CH₂)ₙONH₂, and O(CH₂ₙON(CH₂)nCH₃)₂ where n and m can be from 1 to about 10. Other preferred oligonucleotides comprise one of the following at the 2′ position: C to CO, (lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification comprises 2′-methoxyethoxy (2′-O-CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. A further preferred modification comprises 2′-dimethylaminooxyethoxy, i.e. , a O(CH2)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₂).

Other preferred modifications comprise 2′-methoxy (2′-OCH₃), 2′-aminopropoxy (2′-OCH₂CH₂CH₂NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar.

Oligonucleotides may also comprise nucleobase (often referred to in the art simply as “base”) modifications or substitutions. As used herein, “unmodified” or “natural” nucleobases comprise the purine bases adenine (A) and guanine (G), and the pyrimidine bases thymine (T), cytosine (C) and uracil (U). Modified nucleobases comprise other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituteduracils and cytosines, 7-methylquanine and 7-methyladenine, 8-azaguanine and 8-azaadenine, 7-deazaguanine and 7-deazaadenine and 3-deazaguanine and 3-deazaadenine.

Further, nucleobases comprise those disclosed in U.S. Pat. No. 3,687,808, those disclosed in ‘The Concise Encyclopedia of Polymer Science And Engineering’, pages 858-859, Kroschwitz, J. I., ed. John Wiley & Sons, 1990, those disclosed by Englisch et al., ‘Angewandle Chemie, International Edition’, 1991, 30, page 613, and those disclosed by Sanghvi, Y. S., Chapter 15, ‘Antisense Research and Applications’, pages 289-302, Crooke, S. T. and Lebleu B. ea., CRC Press, 1993. Certain of these nucleobases are particularly useful for increasing the binding affinity of the oligomeric compounds of the invention. These comprise 5-substituted pyrimidines, 6-azapyrimidines and N-2, N-6 and 0-6 substituted purines, comprising 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds, ‘Antisense Research and Applications’, CRC Press, Boca Raton, 1993, pp. 276-278) and are presently preferred base substitutions, even more particularly when combined with 2′-O-methoxyethyl sugar modifications.

Immune System: Immune systems are classified into two general systems, the “innate” or “primary” immune system and the “acquired/adaptive” or “secondary” immune system. It is thought that the innate immune system initially keeps the infection under control, allowing time for the adaptive immune system to develop an appropriate response. Studies have suggested that the various components of the innate immune system trigger and augment the components of the adaptive immune system, including antigen-specific B and T lymphocytes (Kos, Immunol. Res. 1998, 17:303; Romagnani, Immunol. Today. 1992, 13: 379; Banchereau and Steinman, Nature. 1988, 392:245).

A primary immune response refers to an innate immune response that is not affected by prior contact with the antigen. The main protective mechanisms of primary immunity are the skin (protects against attachment of potential environmental invaders), mucous (traps bacteria and other foreign material), gastric acid (destroys swallowed invaders), antimicrobial substances such as interferon (IFN) (inhibits viral replication) and complement proteins (promotes bacterial destruction), fever (intensifies action of interferons, inhibits microbial growth, and enhances tissue repair), natural killer (NK) cells (destroy microbes and certain tumor cells, and attack certain virus infected cells), and the inflammatory response (mobilizes leukocytes such as macrophages and dendritic cells to phagocytose invaders).

Some cells of the innate immune system, including macrophages and dendritic cells (DC), function as part of the adaptive immune system as well by taking up foreign antigens through pattern recognition receptors, combining peptide fragments of these antigens with major histocompatibility complex (MHC) class I and class II molecules, and stimulating naive CD8⁺ and CD4⁺ T cells respectively (Banchereau and Steinman, supra; Holmskov et al., Immunol. Today. 1994, 15:67; Ulevitch and Tobias Anna. Rev. Immunol. 1995, 13:437). Professional antigen-presenting cells (APCs) communicate with these T cells, leading to the differentiation of naive CD4⁺ T cells into T-helper 1 (Thi) or T-helper 2 (Th2) lymphocytes that mediate cellular and humoral immunity, respectively (Trinchieri Annu. Rev. Immunol. 1995, 13:251; Howard and O'Garra, Immunol. Today. 1992, 13:198; Abbas et al., Nature. 1996, 383:787; Okamura et al., Adv. Immunol. 1998, 70:281; Mosmann and Sad, Immunol. Today. 1996, 17:138; O'Garra Immunity. 1998, 8:275).

A secondary immune response or adaptive immune response may be active or passive, and may be humoral (antibody based) or cellular that is established during the life of an animal, is specific for an inducing antigen, and is marked by an enhanced immune response on repeated encounters with said antigen. A key feature of the T lymphocytes of the adaptive immune system is their ability to detect minute concentrations of pathogen-derived peptides presented by MHC molecules on the cell surface. Upon activation, naïve CD4 T cells differentiate into one of at least two cell types, Th1 cells and Th2 cells, each type being characterized by the cytokines it produces. “Th1 cells” are primarily involved in activating macrophages with respect to cellular immunity and the inflammatory response, whereas “Th2 cells” or “helper T cells” are primarily involved in stimulating B cells to produce antibodies (humoral immunity). CD4 is the receptor for the human immunodeficiency virus (HIV). Effector molecules for Th1 cells include, but are not limited to, IFN-γ, GM-CSF, TNF-α, CD40 ligand, Fas ligand, IL-3, TNF-β, and IL-2. Effector molecules for Th2 cells include, but are not limited to, IL-4, IL-5, CD40 ligand, IL-3, GS-CSF, IL-10, TGF-β, and eotaxin. Activation of the Th1 type cytokine response can suppress the Th2 type cytokine response, and reciprocally, activation of the Th2 type cytokine response can suppress the Th1 type response. Thus, the immune response is “polarized” toward a Th1 or Th2 response.

In adaptive immunity, adaptive T and B cell immune responses work together with innate immune responses. The basis of the adaptive immune response is that of clonal recognition and response. An antigen selects the clones of cell which recognize it, and the first element of a specific immune response must be rapid proliferation of the specific lymphocytes. This is followed by further differentiation of the responding cells as the effector phase of the immune response develops. In T-cell mediated non-infective inflammatory diseases and conditions, immunosuppressive drugs inhibit T-cell proliferation and block their differentiation and effector functions.

Characteristics of CD4 T cell subsets: As discussed above, CD4 T cells upon activation and expansion develop into different T helper (T_{H}) cell subsets with different cytokine profiles and distinct effector functions. Appropriate differentiation of T_{H} cells into effector subsets best suited for host defense against an invading pathogen is of critical importance to the immune system. CD4 T cells differentiate into at least four known subsets, three effector subsets (T_{H}1, T_{H}2 and T_{H}17) and one T regulatory subset (Treg). Based on the cytokines that they produce, T cells were historically divided into T_{H}1 and T_{H}2 cells, and this has provided a framework to understand how specific cytokine milieus produced by cells of the innate immune system guide the development of adaptive immunity. T_{H}1 cells, which are potently induced by dendritic cells (DC) secreting IL-12, are characterized by the expression of the lineage-specific transcription factor T-bet (T box 21) and the production of IFN-γ. T_{H}2 cells, which depend on IL-4 during differentiation and lack of IL-12, produce IL-4, IL-5, IL-9, and IL-13 and are characterized by the expression of the transcription factor GATA-3. A third subset of IL-17-producing effector T helper cells, called T_{H}17 cells, has been discovered and characterized and is specified by expression of the transcription factor RORγt.

T_{H}17 cells produce IL-17, IL-17F, and IL-22. By secreting these effector cytokines, T_{H}17 cells induce a massive tissue reaction due to the broad distribution of the IL-17 and IL-22 receptors. T_{H}17 cells also secrete IL-21 to communicate with the cells of the immune system. Synergy between the cytokines transforming growth factor beta isoform 1 (TGF-β) and interleukin (IL)-6 induces development of T_{H}17 cells in mice and humans, while IL-23 supports expansion of these cells. The differentiation factors (TGF-β plus IL-6 or IL-21), the growth and stabilization factor (IL-23), and the transcription factors (STAT3, ROR-γt (ROR-c), and ROR-a) involved in the development of T_{H}17 cells have only recently been identified. The participation of TGF-β in the differentiation of T_{H}17 cells places the T_{H}17 lineage in close relationship with CD4⁺CD25⁺Foxp3⁺ regulatory T cells (T_{reg}) since TGF-β also induces differentiation of naive T cells into Foxp3⁺ Treg in the peripheral immune compartment. Treg cells are a specialized subpopulation of T cells that act to suppress activation of the immune system and thereby maintain immune system homeostasis and tolerance to self-antigens. Development of Treg cells, which are capable of suppressing autoimmune disease, is reciprocally related to T_{H}17 cells, which can drive immune responses, including autoimmune responses. Treg cells can be identified by their unique expression of the transcription factor forkhead box P3 (Foxp3). So far as is known, there are two phenotypically identical populations of CD4^{|}CD25^{|} Treg—natural and adaptive. Natural CD4⁺CD25⁺ Treg cells arise in the thymus under homeostatic conditions to safeguard against autoimmunity Adaptive CD4⁺CD25⁺ Treg cells arise during inflammatory processes such as infections and cancers and suppress immunity through heterogeneous mechanisms that include direct contact or the production of soluble factors such as IL-10 and TGF-β.

In a preferred embodiment, the TNFR25 compositions modulate T cell responses. Preferably, the TNFR25 enhances or up-regulates the T cell response.

In another preferred embodiment, the T cell response is directed to a specific antigen, e.g. viral tumor, bacterial and the like.

The phrase “T cell response” means an immunological response involving T cells. The T cells that are “activated” divide to produce memory T cells or cytotoxic T cells. The cytotoxic T cells bind to and destroy cells recognized as containing the antigen. The memory T cells are activated by the antigen and thus provide a response to an antigen already encountered. This overall response to the antigen is the T cell response.

In another preferred embodiment, the TNFR25 compositions modulate immune cells. Preferably, the TNFR25 compositions increase or enhance the response of the immune cells to a specific antigen, for example, viral antigen, tumor antigen and the like.

“Cells of the immune system” or “immune cells”, is meant to include any cells of the immune system that may be assayed, including, but not limited to, B lymphocytes, also called B cells, T lymphocytes, also called T cells, natural killer (NK) cells, natural killer T (NK) cells, lymphokine-activated killer (LAK) cells, monocytes, macrophages, neutrophils, granulocytes, mast cells, platelets, Langerhan's cells, stem cells, dendritic cells, peripheral blood mononuclear cells, humor-infiltrating (TIL) cells, gene modified immune cells including hybridomas, drug modified immune cells, antigen presenting cells and derivatives, precursors or progenitors of the above cell types.

In another preferred embodiment, the TNFR25 compositions modulate the response of immune effector cells. Preferably, the immune effector cells are up-regulated or enhanced and directed to a specific antigen.

“Immune effector cells” refers to cells, and subsets thereof, e.g. Treg, Th1, Th2, capable of binding an antigen and which mediate an immune response selective for the antigen. These cells include, but are not limited to, T cells (T lymphocytes), B cells (B lymphocytes), antigen presenting cells, such as for example dendritic cells, monocytes, macrophages; myeloid suppressor cells, natural killer (NK) cells and cytotoxic T lymphocytes (CTLs), for example CTL lines, CTL clones, and CTLs from tumor, inflammatory, or other infiltrates.

In another preferred embodiment, the TNFR25 compositions modulate T regulatory cells. Preferably, regulation of Treg cells induces an increase or enhancement of immune cell response to a specific antigen.

Treg cells express the transcription factor Foxp3, which is not upregulated upon T cell activation and discriminates Tregs from activated effector cells. Tregs are identified by the cell surface markers CD25, CD45RB, CTLA4, and GITR. Treg development is induced by MSC activity. Several Treg subsets have been identified that have the ability to inhibit autoimmune and chronic inflammatory responses and to maintain immune tolerance in tumor-bearing hosts. These subsets include interleukin 10-(IL-10-) secreting T regulatory type 1 (Tr1) cells, transforming growth factor-β-(TGF-β-) secreting T helper type 3 (Th3) cells, and “natural” CD4⁺/CD25⁺ Tregs (Trn) (Fehervari and Sakaguchi. J. Clin. Invest. 2004, 114:1209-1217; Chen et al. Science. 1994, 265: 1237-1240; Groux et al. Nature. 1997, 389: 737-742).

The term “myeloid suppressor cell (MSC)” refers to a cell that is of hematopoietic lineage and expresses Gr-1 and CD11b; MSCs are also referred to as immature myeloid cells and were recently renamed to myeloid-derived suppressor cells (MDSCs). MSCs may also express CD115 and/or F4/80 (see Li et al., Cancer Res. 2004, 64:1130-1139). MSCs may also express CD31, c-kit, vascular endothelial growth factor (VEGF)-receptor, or CD40 (Bronte et al., Blood. 2000, 96:3838-3846). MSCs may further differentiate into several cell types, including macrophages, neutrophils, dendritic cells, Langerhan's cells, monocytes or granulocytes. MSCs may be found naturally in normal adult bone marrow of human and animals or in sites of normal hematopoiesis, such as the spleen in newborn mice. Upon distress due to graft-versus-host disease (GVHD), cyclophosphamide injection, or γ-irradiation, for example, MSCs may be found in the adult spleen. MSCs can suppress the immunological response of T cells, induce T regulatory cells, and produce T cell tolerance. Morphologically, MSCs usually have large nuclei and a high nucleus-to-cytoplasm ratio. MSCs can secrete TFG-β and IL-10 and produce nitric oxide (NO) in the presence of IFN-γ or activated T cells. MSCs may form dendriform cells; however, MSCs are distinct from dendritic cells (DCs) in that DCs are smaller and express CD11c; MSCs do not express CD11c. T cell inactivation by MSCs in vitro can be mediated through several mechanisms: IFN-γ-dependent nitric oxide production (Kusmartsev et al. J. Immunol. 2000, 165: 779-785); Th2-mediated-IL-4/IL-13-dependent arginase 1 synthesis (Bronte et al. J. Immunol. 2003, 170: 270-278); loss of CD3ξ signaling in T cells (Rodriguez et al. J. Immunol. 2003, 171: 1232-1239); and suppression of the T cell response through reactive oxygen species (Bronte et al. J Immunol. 2003, 170: 270-278; Bronte et al. Trends Immunol. 2003, 24: 302-306; Kusmartsev et al. J Immunol. 2004, 172: 989-999; Schmielau and Finn, Cancer Res. 2001, 61: 4756-4760).

In another preferred embodiment, modulation of immune cells and subsequent responses comprises a method of treating a patient with a disease such as for example, cancer, viral disease e.g. HIV, or disease caused by any infectious organism wherein an anti-TNFR25 composition, is administered to a patient, and modulates the functions of the immune cells, for example, proliferation of a lymphocyte wherein that lymphocyte had been previously suppressed or attenuated, or in cases where the immune response is normal but the enhancement of the enhancement of the immune response results in more effective and faster treatment of a patient. Negative regulatory pathway, and not lack of inherent tumor immunogenicity (i.e., the ability of the unmanipulated tumors to stimulate protective immunity), play an important role in preventing the immune-mediated control of tumor progression. The therapeutic implication is that countering immune-attenuating/suppressive regulatory circuits contributes to successful immune control of cancer.

In a preferred embodiment, a method of enhancing an immune response to a vaccine comprises administering to a patient in need thereof, a therapeutically effective amount of TNFR25, TNFR25 splice variants, TNFR25-agonists, agonistic anti-TNFR25, TNFR25 antagonists, TNFR25 ligands, variants, mutants or fragments thereof, in conjunction, prior to or after administration of a vaccine or any combination thereof.

In a preferred embodiment, the antigen specific immune response to the vaccine is regulated, preferably up-regulated. The enhancement of the immune response to a vaccine or other antigenic stimulant can be measured by any conventional method, such as for example proliferation assays, cytokine secretion, types of cytokines secreted, cytotoxic T lymphocyte assays, ELISAS, RIA and the like. The enhanced immune response can also be detected by monitoring the treatment. For example, in the case of treating cancer, an enhanced immune response could also be monitored by observing one or more of the following effects: (1) inhibition, to some extent, of tumor growth, including, (i) slowing down (ii) inhibiting angiogenesis and (ii) complete growth arrest; (2) reduction in the number of tumor cells; (3) maintaining tumor size; (4) reduction in tumor size; (5) inhibition, including (i) reduction, (ii) slowing down or (iii) complete prevention, of tumor cell infiltration into peripheral organs; (6) inhibition, including (i) reduction, (ii) slowing down or (iii) complete prevention, of metastasis; (7) enhancement of anti-tumor immune response, which may result in (i) maintaining tumor size, (ii) reducing tumor size, (iii) slowing the growth of a tumor, (iv) reducing, slowing or preventing invasion and/or (8) relief, to some extent, of the severity or number of one or more symptoms associated with the disorder. In the cases of viral infection, plaque assays, viral titers etc., can be used to monitor the clearance of the virus.

In another preferred embodiment, TL1A, TL1A agonists, TL1A antagonists, TL1A ligands, variants, mutants or fragments thereof are optionally administered as part of a treatment regimen or to further modulate the immune response to the vaccine. An example is an HIV vaccine.

In another preferred embodiment, mucosal immune responses are modulated by administration of a composition comprising one or more of: TNFR25, TNFR25 splice variants, TNFR25-agonists, agonistic anti TNFR25,TNFR25 antagonists, TNFR25 ligands, other immune regulating molecules, variants, mutants or fragments.

In another preferred embodiments, cytokines, cell factors, antibodies, such as for example anti-interleukin 4 antibody etc, are optionally administered as part of a treatment regimen.

In another preferred embodiment, a method of modulating an immune response to an antigen in a patient, comprises administering to the patient a therapeutically effective amount of at least one of: TNFR25, TNFR25 splice variants, TNFR25-agonists, agonistic anti TNFR25, TNFR25 antagonists, TNFR25 ligands, TL1A, TL1A ligands, TL1A agonists, TL1A antagonists, variants, mutants or fragments thereof. Optionally, cytokines, growth factors, antibodies or blocking agents, adjuvants, or combinations thereof are optionally administered to the patient. Blocking agents can be directed to one or more cytokines, or other cellular factors depending on the type of immune response is desired. For example, polarizing the immune system toward a Th1 type immune response versus a Th2 type immune response.

In another preferred embodiment, a patient can be can be a responder or non-responder to a particular antigen. For example, some individuals do not mount an immune response to the HBV vaccine or if there is an immune response there is a rapid decrease in detectable anti-HBV antibodies or HBV antigen specific cells when the patient is tested for immunity to HBV antigens.

The immune cells or factors comprising an immune response, include for example, T cells (T lymphocytes), B cells (B lymphocytes), antigen presenting cells, dendritic cells, monocytes, macrophages, myeloid suppressor cells, natural killer (NK) cells, cytotoxic T lymphocytes (CTLs), inflammatory, or other infiltrates and subsets thereof, chemokines, cytokines, antibodies, cell factors, or hormones.

In another preferred embodiment, a method of regulating a mucosal immune response in a patient, comprises administering to a patient, a therapeutically effective amount of at least one of TNFR25, TNFR25 splice variants, TNFR25-agonists, agonistic anti TNFR25,TNFR25 antagonists, TNFR25 ligands, TL1A, TL1A ligands, TL1A agonists, TL1A antagonists, variants, mutants or fragments thereof, or any combination thereof. This is especially important in cases such as for example, HIV whereby the point of entry is usually via mucosa. The regulation of the mucosal immune response is also important in those cases where the immune system is associated with the disease. Examples include, colitis, Crohn's disease, inflammatory bowel diseases, arthritis, autoimmune diseases or disorders, allergies, allergic reactions, asthma, lung inflammation and the like.

The mucosal immune system, consisting of lymphoid tissues associated with the lacrimal, salivary, gastrointestinal, respiratory and urogenital tracts and lactating breasts, quantitatively contains the majority of the lymphoid tissue of the body. There are a number of important features of the gastrointestinal mucosal immune system: the mucosal immune system contains specialized structures, such as the Peyer's patches, where immune responses are likely to be initiated; there is a pattern of relatively specific recirculation of lymphoid cells to the mucosa, known as mucosal homing; subsets of lymphoid cells, particularly IgA B cells and memory T cells, predominate at mucosal surfaces; and the predominant mucosal immunoglobulin, secretory IgA, is particularly well adapted to host defense at mucosal surfaces. These elements of the gastrointestinal mucosal immune system function together to generate an immune response which on the one hand protects the host from harmful pathogens, but on the other hand is tolerant of the ubiquitous dietary antigens and normal microbial flora.

In a preferred embodiment, a method of regulating immune cell activity in vivo, comprises administering to a patient an effective amount of at least one of TNFR25, TNFR25 splice variants, TNFR25-agonists, agonistic anti TNFR25, TNFR25 antagonists, TNFR25 ligands, TL1A, TL1A ligands, TL1A agonists, TL1A antagonists, variants, mutants or fragments thereof, or any combination thereof.

In a preferred embodiment, administration of one or more compositions of the invention to a patient, the immune cell activity is up-regulated or down regulated as compared to a normal control.

In another preferred embodiment, a method of regulating immune cells in vitro comprising culturing cells with at least one of: TNFR25, TNFR25-agonists, agonistic anti TNFR25, TNFR25 antagonists, TNFR25 ligands, TL1A, TL1A ligands, TL1A agonists, TL1A antagonists, variants, mutants or fragments thereof, or any combination thereof.

In another preferred embodiment, a method of polarizing a T lymphocyte helper (Th) response in vivo or in vitro, comprises administering to cells or a patient in need thereof, a therapeutically effective amount of: TNFR25, full length TNFR25, TNFR25 splice variants, TNFR25-agonists, agonistic anti TNFR25, TNFR25 antagonists, TNFR25 ligands, variants, mutants or fragments thereof, or any combination thereof.

In preferred embodiments a T lymphocyte helper 2 (Th2) response is polarized to a T lymphocyte helper 1 (Th1) response. Optionally, other factors which contribute to a Th1 response may be administered during the course of treatment, before, or after administration of one or more compounds of the invention. For example, interleukin-12 (IL-12), interferon gamma (IFN-γ) and/or anti-interleukin 4 (anti-IL-4) antibodies or blocking agents.

Treatments: In a preferred embodiment, a method of treating a disease or disorder associated with an immune response comprises administering to a patient, a therapeutically effective amount of a composition comprising at least one of: TNFR25, TNFR25 splice variants, TNFR25-agonists, agonistic anti TNFR25, TNFR25 antagonists, TNFR25 ligands, TL1A, TL1 A ligands, TL agonists, TL antagonists, variants, mutants or fragments thereof.

In a preferred embodiment, a disease or disorder associated with an immune response comprises: autoimmunity, inflammation, allergies, asthma, colitis, multiple sclerosis, Crohn's disease, irritable bowel syndrome, or arthritis. Other examples include, Such diseases or disorders comprise, for example: rejection reactions by transplantation of organs or tissues such as the heart, kidney, liver, bone marrow, skin, cornea, lung, pancreas, small intestine, limb, muscle, nerve, intervertebral disc, trachea, myoblast, cartilage, etc.; graft-versus-host reactions following bone marrow transplantation; autoimmune diseases such as rheumatoid arthritis, systemic lupus erythematosus, Hashimoto's thyroiditis, multiple sclerosis, myasthenia gravis, type I diabetes, etc.; infections caused by pathogenic microorganisms (e.g. Aspergillus fumigatus, Fusarium oxysporum, Trichophyton asteroides, etc.); inflammatory or hyperproliferative skin diseases or cutaneous manifestations of immunologically mediated diseases (e.g. psoriasis, atopic dermatitis, contact dermatitis, eczematoid dermatitis, seborrheic dermatitis, lichen planus, pemphigus, bullous pemphigoid, epidermolysis bullosa, urticaria, angioedema, vasculitides, erythema, dermal eosinophilia, lupus erythematosus, acne, and alopecia areata); autoimmune diseases of the eye (e.g. keratoconjunctivitis, vernal conjunctivitis, uveitis associated with Behcet's disease, keratitis, herpetic keratitis, conical keratitis, corneal epithelial dystrophy, keratoleukoma, ocular premphigus, Mooren's ulcer, scleritis, Graves' ophthalmopathy, Vogt-Koyanagi-Harada syndrome, keratoconjunctivitis sicca (dry eye), phlyctenule, iridocyclitis, sarcoidosis, endocrine ophthalmopathy, etc.); reversible obstructive airways diseases [asthma (e.g. bronchial asthma, allergic asthma, intrinsic asthma, extrinsic asthma, and dust asthma), particularly chronic or inveterate asthma (e.g. late asthma and airway hyper-responsiveness) bronchitis, etc.; mucosal or vascular inflammations (e.g. gastric ulcer, ischemic or thrombotic vascular injury, ischemic bowel diseases, enteritis, necrotizing enterocolitis, intestinal damages associated with thermal burns, leukotriene B4-mediated diseases); intestinal inflammations/allergies (e.g. coeliac diseases, proctitis, eosinophilic gastroenteritis, mastocytosis, Crohn's disease and ulcerative colitis); food-related allergic diseases with symptomatic manifestation remote from the gastrointestinal tract (e.g. migrain, rhinitis and eczema); renal diseases (e.g. intestitial nephritis, Goodpasture's syndrome, hemolytic uremic syndrome, and diabetic nephropathy); nervous diseases (e.g. multiple myositis, Guillain-Barre syndrome, Meniere's disease, multiple neuritis, solitary neuritis, cerebral infarction, Alzheimer's diseases Parkinson's diseases, amyotrophic lateral sclerosis (ALS) and radiculopathy); cerebral ischemic disease (e.g., head injury, hemorrhage in brain (e.g., subarachnoid hemorrhage, intracerebral hemorrhage), cerebral thrombosis, cerebral embolism, cardiac arrest, stroke, transient ischemic attack (TIA), hypertensive encephalopathy, cerebral infarction); endocrine diseases (e.g. hyperthyroidism, and Basedow's disease); hematic diseases (e.g. pure red cell aplasia, aplastic anemia, hypoplastic anemia, idiopathic thrombocytopenic purpura, autoimmune hemolytic anemia, agranulocytosis, pernicious anemia, megaloblastic anemia, and anerythroplasia); bone diseases (e.g. osteoporosis); respiratory diseases (e.g. sarcoidosis, pulmonary fibrosis, and idiopathic interstitial pneumonia); skin diseases (e.g. dermatomyositis, leukoderma vulgaris, ichthyosis vulgaris, photosensitivity, and cutaneous T-cell lymphoma); circulatory diseases (e.g. arteriosclerosis, atherosclerosis, aortitis syndrome, polyarteritis nodosa, and myocardosis); collagen diseases (e.g. scleroderma, Wegener's granuloma, and Sjogren's syndrome); adiposis; eosinophilic fasciitis; periodontal diseases (e.g. damage to gingiva, periodontium, alveolar bone or substantia ossea dentis); nephrotic syndrome (e.g. glomerulonephritis); male pattern alopecia, alopecia senile; muscular dystrophy; pyoderma and Sezary syndrome; chromosome abnormality-associated diseases (e.g. Down's syndrome); Addison's disease; active oxygen-mediated diseases [e.g. organ injury (e.g. ischemic circulation disorders of organs (e.g. heart, liver, kidney, digestive tract, etc.) associated with preservation, transplantation, or ischemic diseases (e.g. thrombosis, cardial infarction, etc.)); intestinal diseases (e.g. endotoxin shock, pseudomembranous colitis, and drug- or radiation-induced colitis); renal diseases (e.g. ischemic acute renal insufficiency, chronic renal failure); pulmonary diseases (e.g. toxicosis caused by pulmonary oxygen or drugs (e.g. paracort, bleomycin, etc.), lung cancer, and pulmonary emphysema); ocular diseases (e.g. cataracta, iron-storage disease (siderosis bulbi), retinitis, pigmentosa, senile plaques, vitreous scarring, corneal alkali burn); dermatitis (e.g. erythema multiforme, linear immunoglobulin A bullous dermatitis, cement dermatitis); and other diseases (e.g. gingivitis, periodontitis, sepsis, pancreatitis, and diseases caused by environmental pollution (e.g. air pollution), aging, carcinogen, metastasis of carcinoma, and hypobaropathy)]; diseases caused by histamine release or leukotriene C4 release; restenosis of coronary artery following angioplasty and prevention of postsurgical adhesions; autoimmune diseases and inflammatory conditions (e.g., primary mucosal edema, autoimmune atrophic gastritis, premature menopause, male sterility, juvenile diabetes mellitus, pemphigus vulgaris, pemphigoid, sympathetic ophthalmitis, lens-induced uveitis, idiopathic leukopenia, active chronic hepatitis, idiopathic cirrhosis, discoid lupus erythematosus, autoimmune orchitis, arthritis (e.g. arthritis deformans), or polychondritis); Human Immunodeficiency Virus (HIV) infection, AIDS; allergic conjunctivitis; hypertrophic cicatrix and keloid due to trauma, burn, or surgery.

### Formulations

The invention contemplates delivery of nucleic acids, polypeptides, peptides, vectors, cells comprising TNFR25 nucleic acids or polypeptides, of the TNFR25 compositions, for example, full length TNFR25; TNFR25 splice variants and the like. Delivery of polypeptides and peptides can be accomplished according to standard vaccination protocols which are well known in the art.

In another preferred embodiment, a vector comprises a TNFR25 polynucleotide, natural splice variants, deletions, variants, mutants or active fragments thereof.

The vector can be administered to a patient wherein expression of TNFR25, variants, mutants or active fragments thereof, induces a response comprising at least one of: anti-viral activity, immune responses, immune signaling or intracellular B-form DNA.

A number of vectors are known to be capable of mediating transfer of gene products to mammalian cells, as is known in the art and described herein. A “vector” (sometimes referred to as gene delivery or gene transfer “vehicle”) refers to a macromolecule or complex of molecules comprising a polynucleotide to be delivered to a host cell, either in vitro or in vivo. The polynucleotide to be delivered may comprise a coding sequence of interest in gene therapy. Vectors include, for example, viral vectors (such as adenoviruses (“Ad”), adeno-associated viruses (AAV), and vesicular stomatitis virus (VSV) and retroviruses), liposomes and other lipid-containing complexes, and other macromolecular complexes capable of mediating delivery of a polynucleotide to a host cell. Vectors can also comprise other components or functionalities that further modulate gene delivery and/or gene expression, or that otherwise provide beneficial properties to the targeted cells. As described and illustrated in more detail below, such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Other vectors include those described by Chen et al; BioTechniques, 34: 167-171 (2003). A large variety of such vectors are known in the art and are generally available.

A “recombinant viral vector” refers to a viral vector comprising one or more heterologous gene products or sequences. Since many viral vectors exhibit size-constraints associated with packaging, the heterologous gene products or sequences are typically introduced by replacing one or more portions of the viral genome. Such viruses may become replication-defective, requiring the deleted function(s) to be provided in trans during viral replication and encapsidation (by using, e.g., a helper virus or a packaging cell line carrying gene products necessary for replication and/or encapsidation). Modified viral vectors in which a polynucleotide to be delivered is carried on the outside of the viral particle have also been described (see, e.g., Curiel, D T, et al. PNAS 88: 8850-8854, 1991).

Suitable nucleic acid delivery systems include viral vector, typically sequence from at least one of an adenovirus, adenovirus-associated virus (AAV), helper-dependent adenovirus, retrovirus, or hemagglutinating virus of Japan-liposome (HVJ) complex. Preferably, the viral vector comprises a strong eukaryotic promoter operably linked to the polynucleotide e.g., a cytomegalovirus (CMV) promoter.

Additionally preferred vectors include viral vectors, fusion proteins and chemical conjugates. Retroviral vectors include Moloney murine leukemia viruses and HIV-based viruses. One preferred HIV-based viral vector comprises at least two vectors wherein the gag and pol genes are from an HIV genome and the env gene is from another virus. DNA viral vectors are preferred. These vectors include pox vectors such as orthopox or avipox vectors, herpesvirus vectors such as a herpes simplex I virus (HSV) vector [Geller, A. I. et al., J. Neurochem, 64: 487 (1995); Lim, F., et al., in DNA Cloning: Mammalian Systems, D. Glover, Ed. (Oxford Univ. Press, Oxford England) (1995); Geller, A. I. et al., Proc Natl. Acad. Sci.: U.S.A.:90 7603 (1993); Geller, A. I., et al., Proc Natl. Acad. Sci USA: 87:1149 (1990)], Adenovirus Vectors [LeGal LaSalle et al., Science, 259:988 (1993); Davidson, et al., Nat. Genet. 3: 219 (1993); Yang, et al., J. Virol. 69: 2004 (1995)] and Adeno-associated Virus Vectors [Kaplitt, M. G., et al., Nat. Genet. 8:148 (1994)].

Pox viral vectors introduce the gene into the cells cytoplasm. Avipox virus vectors result in only a short term expression of the nucleic acid. Adenovirus vectors, adeno-associated virus vectors and herpes simplex virus (HSV) vectors may be an indication for some invention embodiments. The adenovirus vector results in a shorter term expression (e.g., less than about a month) than adeno-associated virus, in some embodiments, may exhibit much longer expression. The particular vector chosen will depend upon the target cell and the condition being treated. The selection of appropriate promoters can readily be accomplished. Preferably, one would use a high expression promoter. An example of a suitable promoter is the 763-base-pair cytomegalovirus (CMV) promoter. The Rous sarcoma virus (RSV) (Davis, et al., Hum Gene Ther 4:151 (1993)) and MMT promoters may also be used. Certain proteins can expressed using their native promoter. Other elements that can enhance expression can also be included such as an enhancer or a system that results in high levels of expression such as a tat gene and tar element. This cassette can then be inserted into a vector, e.g., a plasmid vector such as, pUC19, pUC118, pBR322, or other known plasmid vectors, that includes, for example, an E. coli origin of replication. See, Sambrook, et al., Molecular Cloning: A Laboratory Manual, Cold Spring Harbor Laboratory press, (1989). The plasmid vector may also include a selectable marker such as the β-lactamase gene for ampicillin resistance, provided that the marker polypeptide does not adversely effect the metabolism of the organism being treated. The cassette can also be bound to a nucleic acid binding moiety in a synthetic delivery system, such as the system disclosed in WO 95/22618.

If desired, the polynucleotides of the invention may also be used with a microdelivery vehicle such as cationic liposomes and adenoviral vectors. For a review of the procedures for liposome preparation, targeting and delivery of contents, see Mannino and Gould-Fogerite, BioTechniques, 6:682 (1988). See also, Felgner and Holm, Bethesda Res. Lab. Focus, 11(2):21 (1989) and Maurer, R. A., Bethesda Res. Lab. Focus, 11(2):25 (1989).

Replication-defective recombinant adenoviral vectors, can be produced in accordance with known techniques. See, Quantin, et al., Proc. Natl. Acad. Sci. USA, 89:2581-2584 (1992); Stratford-Perricadet, et al., J. Clin. Invest., 90:626-630 (1992); and Rosenfeld, et al., Cell, 68:143-155 (1992).

Another delivery method is to use single stranded DNA producing vectors which can produce the TNFR25 intracellularly. See for example, Chen et al, BioTechniques, 34: 167-171 (2003), which is incorporated herein, by reference, in its entirety.

Expression of TNFR25 molecules may be controlled by any promoter/enhancer element known in the art, but these regulatory elements must be functional in the host selected for expression. Promoters which may be used to control TNFR25 gene expression include, but are not limited to, cytomegalovirus (CMV) promoter (U.S. Pat. Nos. 5,385,839 and 5,168,062), the SV40 early promoter region (Benoist and Chambon, 1981, Nature 290:304-310), the promoter contained in the 3′ long terminal repeat of Rous sarcoma virus (Yamamoto, et al., Cell 22:787-797, 1980), the herpes thymidine kinase promoter (Wagner et al., Proc. Natl. Acad. Sci. U.S.A. 78:1441-1445, 1981), the regulatory sequences of the metallothionein gene (Brinster et al., Nature 296:39-42, 1982); prokaryotic expression vectors such as the β-lactamase promoter (Villa-Kamaroff, et al., Proc. Natl. Acad. Sci. U.S.A. 75:3727-3731, 1978), or the kw promoter (DeBoer, et al., Proc. Natl. Acad. Sci. U.S.A. 80:21-25, 1983); see also “Useful proteins from recombinant bacteria” in Scientific American, 242:74-94, 1980; promoter elements from yeast or other fungi such as the Gal 4 promoter, the ADC (alcohol dehydrogenase) promoter, PGK (phosphoglycerol kinase) promoter, alkaline phosphatase promoter; and the animal transcriptional control regions, which exhibit tissue specificity and have been utilized in transgenic animals: elastase I gene control region which is active in pancreatic acinar cells (Swift et al., Cell 38:639-646, 1984; Ornitz et al., Cold Spring Harbor Symp. Quant. Biol. 50:399-409, 1986; MacDonald, Hepatology 7:425-515 1987); insulin gene control region which is active in pancreatic beta cells (Hanahan, Nature 315:115-122, 1985), immunoglobulin gene control region which is active in lymphoid cells (Grosschedl et al., Cell 38:647-658, 1984; Adames et al., Nature 318:533-538, 1985; Alexander et al., Mol. Cell. Biol. 7:1436-1444, 1987), mouse mammary tumor virus control region which is active in testicular, breast, lymphoid and mast cells (Leder et al., Cell 45:485-495, 1986), albumin gene control region which is active in liver (Pinkert et al., Genes and Devel. 1:268-276, 1987), alpha-fetoprotein gene control region which is active in liver (Krumlauf et al., Mol. Cell. Biol. 5:1639-1648, 1985; Hammer et al., Science 235:53-58, 1987), alpha 1-antitrypsin gene control region which is active in the liver (Kelsey et al., Genes and Devel. 1: 161-171, 1987), beta-globin gene control region which is active in myeloid cells (Mogram et al., Nature 315:338-340, 1985; Kollias et al., Cell 46:89-94, 1986), myelin basic protein gene control region which is active in oligodendrocyte cells in the brain (Readhead et al., Cell 48:703-712, 1987), myosin light chain-2 gene control region which is active in skeletal muscle (Sani, Nature 314:283-286, 1985), and gonadotropic releasing hormone gene control region which is active in the hypothalamus (Mason et al., Science 234:1372-1378, 1986).

A wide variety of host/expression vector combinations may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids co/E1, pCR1, pBR322, pMal-C2, pET, pGEX (Smith et al., Gene 67:31-40, 1988), pMB9 and their derivatives, plasmids such as RP4; phage DNAs, e.g., the numerous derivatives of phage 1, e.g., NM989, and other phage DNA, e.g., M13 and filamentous single stranded phage DNA; yeast plasmids such as the 2μ plasmid or derivatives thereof, vectors useful in eukaryotic cells, such as vectors useful in insect or mammalian cells; vectors derived from combinations of plasmids and phage DNAs, such as plasmids that have been modified to employ phage DNA or other expression control sequences; and the like.

Yeast expression systems can also be used according to the invention to express TNFR25. For example, the non-fusion pYES2 vector (XbaI, SphI, ShoI, NotI, GstXI, EcoRI, BstXI, BamH1, SacI, Kpn1, and HindIII cloning sites; Invitrogen) or the fusion pYESHisA, B, C (XbaI, SphI, ShoI, NotI, BstXI, EcoRI, BamHl, SacI, KpnI, and HindIII cloning sites, N-terminal peptide purified with ProBond resin and cleaved with enterokinase; Invitrogen), to mention just two, can be employed according to the invention. A yeast two-hybrid expression system can be prepared in accordance with the invention.

One preferred delivery system is a recombinant viral vector that incorporates one or more of the polynucleotides therein, preferably about one polynucleotide. Preferably, the viral vector used in the invention methods has a pfu (plague forming units) of from about 108 to about 5×1010 pfu. In embodiments in which the polynucleotide is to be administered with a non-viral vector, use of between from about 0.1 nanograms to about 4000 micrograms will often be useful e.g., about 1 nanogram to about 100 micrograms

In a preferred embodiment, a composition of the invention is administered to a patient via immunization routes. For example, intra-venously, intra-muscularly, intra-peritoneally, and the like.

In the case of polynucleotides or oligonucleotides, the delivery of the nucleic acid, e.g. encoding TNFR25 splice variant, can be accomplished by ex vivo methods, i.e. by removing a cell from a subject, genetically engineering the cell to include the nucleic acid, and reintroducing the engineered cell into the subject. One example of such a procedure is outlined in U.S. Pat. No. 5,399,346. In general, it involves introduction in vitro of a functional copy of a gene into a cell(s) of a subject, and returning the genetically engineered cell(s) to the subject. The functional copy of the gene is under operable control of regulatory elements which permit expression of the gene in the genetically engineered cell(s). Numerous transfection and transduction techniques as well as appropriate expression vectors are well known to those of ordinary skill in the art, some of which are described in PCT application WO95/00654. In vivo nucleic acid delivery using vectors such as viruses and targeted liposomes also is contemplated according to the invention.

In another preferred embodiment, an isolated cell expresses TNFR25 or TNFR25 splice variants. The cell can be autologous, syngeneic, xenogeneic et, stem cell, immune cell, mucosal cell and the like.

In another embodiment, the TNFR25 compositions can be administered to autologous cells, allow the cells to expand and then re-infuse the cells into the patient.

The TNFR25 compositions can be administered in a pharmaceutical composition, as a polynucleotide in a vector, liposomes, nucleic acids peptides and the like.

In another preferred embodiment, the TNFR25 compositions can be administered with one or more or additional pharmacologically active agents. As used herein, the term “ pharmacologically active agent” refers to any agent, such as a drug, capable of having a physiologic effect (e.g., a therapeutic or prophylactic effect) on prokaryotic or eukaryotic cells, in vivo or in vitro, including, but without limitation, chemotherapeutics, toxins, radiotherapeutics, radiosensitizing agents, gene therapy vectors, antisense nucleic acid constructs or small interfering RNA, imaging agents, diagnostic agents, agents known to interact with an intracellular protein, polypeptides, and polynucleotides.

The additional pharmacologically active agent can be selected from a variety of known classes of drugs, including, for example, analgesics, anesthetics, anti-inflammatory agents, anthelmintics, anti-arrhythmic agents, antiasthma agents, antibiotics (including penicillins), anticancer agents (including Taxol), anticoagulants, antidepressants, antidiabetic agents, antiepileptics, antihistamines, antitussives, antihypertensive agents, antimuscarinic agents, antimycobacterial agents, antineoplastic agents, antioxidant agents, antipyretics, immunosuppressants, immunostimulants, antithyroid agents, antiviral agents, anxiolytic sedatives (hypnotics and neuroleptics), astringents, bacteriostatic agents, beta-adrenoceptor blocking agents, blood products and substitutes, bronchodilators, buffering agents, cardiac inotropic agents, chemotherapeutics, contrast media, corticosteroids, cough suppressants (expectorants and mucolytics), diagnostic agents, diagnostic imaging agents, diuretics, dopaminergics (antiparkinsonian agents), free radical scavenging agents, growth factors, haemostatics, immunological agents, lipid regulating agents, muscle relaxants, proteins, peptides and polypeptides, parasympathomimetics, parathyroid calcitonin and biphosphonates, prostaglandins, radio-pharmaceuticals, hormones, sex hormones (including steroids), time release binders, anti-allergic agents, stimulants and anoretics, steroids, sympathomimetics, thyroid agents, vaccines, vasodilators, and xanthines.

The additional pharmacologically active agent need not be a therapeutic agent. For example, the agent may be cytotoxic to the local cells to which it is delivered but have an overall beneficial effect on the subject. Further, the agent may be a diagnostic agent with no direct therapeutic activity per se, such as a contrast agent for bioimaging.

In another preferred embodiment, a TNFR25 polynucleotide or peptide are labeled with a detectable marker, such as for example, fluorescent markers (e.g. GFP, RFP etc) or radiolabels.

In another preferred embodiment, the TNFR25 agents are fused with one or more moieties. For example, one or more TNFR25 nucleic acids, proteins or peptides can be linked or fused to another moiety. For example, a targeting sequence, such as, for example, an aptamer, antibody sequence; a therapeutic effector molecule, e.g. cytokines, antibiotics, toxins, radiolabels; signal leader peptide; intracellular targeting moiety and the like. The TNFR25 agents can be genetically fused, or linked via linker molecules.

In another preferred embodiment, the molecule comprises a label for detecting the fusion molecule in vivo and to monitor the effects of the chimeric molecule during therapy.

“Detectable moiety” or a “label” refers to a composition detectable by spectroscopic, photochemical, biochemical, immunochemical, or chemical means. For example, useful labels include ³²P, ³⁵S, fluorescent dyes, electron-dense reagents, enzymes (e.g., as commonly used in an ELISA), biotin-streptavidin, dioxigenin, haptens and proteins for which antisera or monoclonal antibodies are available, or nucleic acid molecules with a sequence complementary to a target. The detectable moiety often generates a measurable signal, such as a radioactive, chromogenic, or fluorescent signal, that can be used to quantify the amount of bound detectable moiety in a sample. Quantitation of the signal is achieved by, e.g., scintillation counting, densitometry, or flow cytometry.

In another preferred embodiment, TNFR25 agents can be radiolabeled. Uses include therapeutic and imaging for diagnostic purposes. The label may be a radioactive atom, an enzyme, or a chromophore moiety. Methods for labeling antibodies have been described, for example, by Hunter and Greenwood, Nature, 144:945 (1962) and by David et al. Biochemistry 13:1014-1021 (1974). Additional methods for labeling antibodies have been described in U.S. Pat. Nos. 3,940,475 and 3,645,090. Methods for labeling oligonucleotide probes have been described, for example, by Leary et al. Proc. Natl. Acad. Sci. USA (1983) 80:4045; Renz and Kurz, Nucl. Acids Res. (1984) 12:3435; Richardson and Gumport, Nucl. Acids Res. (1983) 11:6167; Smith et al. Nucl. Acids Res. (1985) 13:2399; and Meinkoth and Wahl, Anal. Biochem. (1984) 138:267.

The label may be radioactive. Some examples of useful radioactive labels include ³²I, ¹²⁵I, ¹³¹I, and ³H. Use of radioactive labels have been described in U.K. 2,034,323, U.S. Pat. No. 4,358,535, and U.S. Pat. No. 4,302,204.

Some examples of non-radioactive labels include enzymes, chromophores, atoms and molecules detectable by electron microscopy, and metal ions detectable by their magnetic properties.

Some useful enzymatic labels include enzymes that cause a detectable change in a substrate. Some useful enzymes and their substrates include, for example, horseradish peroxidase (pyrogallol and o-phenylenediamine), β-galactosidase (fluorescein β-D-galactopyranoside), and alkaline phosphatase (5-bromo-4-chloro-3-indolylphosphate/nitro blue tetrazolium). The use of enzymatic labels has been described in U.K. 2,019,404, EP 63,879, and by Rotman, Proc. Natl. Acad. Sci. USA, 47, 1981-1991 (1961).

Useful chromophores include, for example, fluorescent, chemiluminescent, and bioluminescent molecules, as well as dyes. Some specific chromophores useful in the present invention include, for example, fluorescein, rhodamine Texas red, phycoerythrin, umbelliferone, luminol.

The labels may be conjugated to the antibody or nucleotide probe by methods that are well known in the art. The labels may be directly attached through a functional group on the probe. The probe either contains or can be caused to contain such a functional group. Some examples of suitable functional groups include, for example, amino, carboxyl, sulfhydryl, maleimide, isocyanate, isothiocyanate. Alternatively, labels such as enzymes and chromophores may be conjugated to the antibodies or nucleotides by means of coupling agents, such as dialdehydes, carbodiimides, dimaleimides, and the like.

The label may also be conjugated to the probe by means of a ligand attached to the probe by a method described above and a receptor for that ligand attached to the label. Any of the known ligand-receptor combinations is suitable. Some suitable ligand-receptor pairs include, for example, biotin-avidin or biotin-streptavidin, and antibody-antigen.

In another preferred embodiment, the TNFR25 molecules of the invention can be used for imaging. In imaging uses, the complexes are labeled so that they can be detected outside the body. Typical labels are radioisotopes, usually ones with short half-lives. The usual imaging radioisotopes, such as ¹²³I, ¹²⁴I, ¹²⁵I, ¹³¹I, ^{99m}TC, ¹⁸⁶Re, ¹⁸⁸Re, ⁶⁴Cu, ⁶⁷Cu, ²¹²Bi, ²¹³Bi, ⁶⁷Ga, ⁹⁰Y, ¹¹¹In, ¹⁸F, ³H, ¹⁴C, ³⁵S or ³²P can be used. Nuclear magnetic resonance (NMR) imaging enhancers, such as gadolinium-153, can also be used to label the complex for detection by NMR. Methods and reagents for performing the labeling, either in the polynucleotide or in the protein moiety, are considered known in the art.

In another preferred embodiment, antibodies are generated, both polyclonal or monoclonal to TNFR25 agents.

In another preferred embodiment, an aptamer is specific for a TNFR25 agent. As used herein, the term “aptamer” or “selected nucleic acid binding species” shall include non-modified or chemically modified RNA or DNA. The method of selection may be by, but is not limited to, affinity chromatography and the method of amplification by reverse transcription (RT) or polymerase chain reaction (PCR).

### Administration of Compositions

The compositions of the present invention may be administered in conjunction with one or more additional active ingredients, pharmaceutical compositions, or vaccines. The therapeutic agents of the present invention may be administered to an animal, preferably a mammal, most preferably a human.

The pharmaceutical formulations and vaccines may be for administration by oral (solid or liquid), parenteral (intramuscular, intraperitoneal, intravenous (IV) or subcutaneous injection), transdermal (either passively or using ionophoresis or electroporation), transmucosal (nasal, vaginal, rectal, or sublingual), or inhalation routes of administration, or using bioerodible inserts and can be formulated in dosage forms appropriate for each route of administration.

The TNFR25 agents may be formulated in pharmaceutically acceptable carriers or diluents such as physiological saline or a buffered salt solution. Suitable carriers and diluents can be selected on the basis of mode and route of administration and standard pharmaceutical practice. A description of exemplary pharmaceutically acceptable carriers and diluents, as well as pharmaceutical formulations, can be found in Remington's Pharmaceutical Sciences, a standard text in this field, and in USP/NF. Other substances may be added to the compositions to stabilize and/or preserve the compositions.

The compositions of the invention may be administered to animals by any conventional technique. The compositions may be administered directly to a target site by, for example, surgical delivery to an internal or external target site, or by catheter to a site accessible by a blood vessel. Other methods of delivery, e.g., liposomal delivery or diffusion from a device impregnated with the composition, are known in the art. The compositions may be administered in a single bolus, multiple injections, or by continuous infusion (e.g., intravenously). For parenteral administration, the compositions are preferably formulated in a sterilized pyrogen-free form.

In some embodiments, the compositions or vaccines are administered by pulmonary delivery. The composition or vaccine is delivered to the lungs of a mammal while inhaling and traverses across the lung epithelial lining to the blood stream [see, e.g., Adjei, et al. Pharmaceutical Research 1990; 7:565 569; Adjei, et al. Int. J. Pharmaceutics 1990; 63:135 144 (leuprolide acetate); Braquet, et al. J. Cardiovascular Pharmacology 1989;13(sup5):143 146 (endothelin-1); Hubbard, et al. (1989) Annals of Internal Medicine, Vol. III, pp. 206 212 (al antitrypsin); Smith, et al. J. Clin. Invest. 1989;84:1145-1146 (α 1-proteinase); Oswein, et al. “Aerosolization of Proteins”, 1990; Proceedings of Symposium on Respiratory Drug Delivery II Keystone, Colorado (recombinant human growth hormone); Debs, et al. J. Immunol. 1988;140:3482 3488 (interferon γ and tumor necrosis factor α); and U.S. Pat. No. 5,284,656 to Platz, et al. (granulocyte colony stimulating factor). A method and composition for pulmonary delivery of drugs for systemic effect is described in U.S. Pat. No. 5,451,569 to Wong, et al. See also U.S. Pat. No. 6,651,655 to Licalsi et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art. Some specific examples of commercially available devices suitable for the practice of this invention are the Ultravent nebulizer (Mallinckrodt Inc., St. Louis, Mo.); the Acorn II nebulizer (Marquest Medical Products, Englewood, Colo.); the Ventolin metered dose inhaler (Glaxo Inc., Research Triangle Park, N.C.); and the Spinhaler powder inhaler (Fisons Corp., Bedford, Mass.). All such devices require the use of formulations suitable for the dispensing of the therapeutic agent. Typically, each formulation is specific to the type of device employed and may involve the use of an appropriate propellant material, in addition to the usual diluents, adjuvants, surfactants and/or carriers useful in therapy. Also, the use of liposomes, microcapsules or microspheres, inclusion complexes, or other types of carriers is contemplated.

Formulations for use with a metered dose inhaler device will generally comprise a finely divided powder containing the therapeutic agent suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2 tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing the therapeutic agent, and may also include a bulking agent, such as lactose, sorbitol, sucrose, or mannitol in amounts which facilitate dispersal of the powder from the device, e.g., 50 to 90% by weight of the formulation. The therapeutic agent should most advantageously be prepared in particulate form with an average particle size of less than 10 mm (or microns), most preferably 0.5 to 5 mm, for most effective delivery to the distal lung.

Nasal or other mucosal delivery of the therapeutic agent is also contemplated. Nasal delivery allows the passage to the blood stream directly after administering the composition to the nose, without the necessity for deposition of the product in the lung. Formulations for nasal delivery include those with dextran or cyclodextran and saponin as an adjuvant.

Methods of Stimulating an Immune Response: In a typical immunization regime employing the TNFR25 compositions of the present invention, the formulations may be administered in several doses (e.g. 1-4). The dose will be determined by the immunological activity the composition produced and the condition of the patient, as well as the body weight or surface areas of the patient to be treated. The size of the dose also will be determined by the existence, nature, and extent of any adverse side effects that may accompany the administration of a particular composition in a particular patient.

The compositions of the present invention may be administered via a non-mucosal or mucosal route. These administrations may include in vivo administration via parenteral injection (e.g. intravenous, subcutaneous, and intramuscular) or other traditional direct routes, such as buccal/sublingual, rectal, oral, nasal, topical (such as transdermal and ophthalmic), vaginal, pulmonary, intraarterial, intraperitoneal, intraocular, or intranasal routes or directly into a specific tissue. Alternatively, the compositions of the invention may be administered by any of a variety of routes such as oral, topical, subcutaneous, mucosal, intravenous, intramuscular, intranasal, sublingual, transcutaneous, subdermal, intradermal and via suppository. Administration may be accomplished simply by direct administration using a patch, needle, catheter or related device, at a single time point or at multiple time points.

Immunization via the mucosal surfaces offers numerous potential advantages over other routes of immunization. The most obvious benefits are 1) mucosal immunization does not require needles or highly-trained personnel for administration, and 2) immune responses are raised at the site(s) of pathogen entry, as well as systemically (Isaka et al. 1999; Kozlowski et al. 1997; Mestecky et al. 1997; Wu et al. 1997).

Extended release systems: A first extended release system includes matrix systems, in which the agent is embedded or dispersed in a matrix of another material that serves to retard the release of the agent into an aqueous environment (i.e., the luminal fluid of the GI tract). When the agent is dispersed in a matrix of this sort, release of the drug takes place principally from the surface of the matrix. Thus the drug is released from the surface of a device, which incorporates the matrix after it diffuses through the matrix or when the surface of the device erodes, exposing the drug. In some embodiments, both mechanisms can operate simultaneously. The matrix systems may be large, i.e., tablet sized (about 1 cm), or small (<0.3 cm). The system may be unitary (e.g., a bolus), may be divided by virtue of being composed of several sub-units (for example, several capsules which constitute a single dose) which are administered substantially simultaneously, or may comprise a plurality of particles, also denoted a multiparticulate. A multiparticulate can have numerous formulation applications. For example, a multiparticulate may be used as a powder for filling a capsule shell, or used per se for mixing with food to ease the intake.

In a specific embodiment, a matrix multiparticulate, comprises a plurality of the agent-containing particles, each particle comprising the agent and/or an analogue thereof e.g. in the form of a solid solution/dispersion with one or more excipients selected to form a matrix capable of controlling the dissolution rate of the agent into an aqueous medium. The matrix materials useful for this embodiment are generally hydrophobic materials such as waxes, some cellulose derivatives, or other hydrophobic polymers. If needed, the matrix materials may optionally be formulated with hydrophobic materials, which can be used as binders or as enhancers. Matrix materials useful for the manufacture of these dosage forms such as: ethylcellulose, waxes such as paraffin, modified vegetable oils, camauba wax, hydrogenated castor oil, beeswax, and the like, as well as synthetic polymers such as poly(vinyl chloride), poly(vinyl acetate), copolymers of vinyl acetate and ethylene, polystyrene, and the like. Water soluble or hydrophilic binders or release modifying agents which can optionally be formulated into the matrix include hydrophilic polymers such as hydroxypropyl cellulose (HPC), hydroxypropyl methyl cellulose (HPMC), methyl cellulose, poly (N-vinyl-2-pyrrolidinone) (PVP), polyethylene oxide) (PEO), poly(vinyl alcohol) (PVA), xanthan gum, carrageenan, and other such natural and synthetic materials. In addition, materials, which function as release-modifying agents include water-soluble materials such as sugars or salts. Preferred water-soluble materials include lactose, sucrose, glucose, and mannitol, as well as hydrophilic polymers like e.g. HPC, HPMC, and PVP.

In a specific embodiment, a multiparticulate product is defined as being processed by controlled agglomeration. In this case the agent is dissolved or partly dissolved in a suitable meltable carrier and sprayed on carrier particles comprising the matrix substance.

Dose: An effective dose of a composition of the presently disclosed subject matter is administered to a subject in need thereof. A “therapeutically effective amount” or a “therapeutic amount” is an amount of a therapeutic composition sufficient to produce a measurable response (e.g., a biologically or clinically relevant response in a subject being treated). For example, in the case of a polarized Th cell response, an effective amount is the lowest amount of an agent necessary to polarize an immune response from a Th2 to Th1 and vice versa. The response can be measured in many ways, as discussed above, e.g. cytokine profiles, cell types, cell surface molecules, etc. Actual dosage levels of active ingredients in the compositions of the presently disclosed subject matter can be varied so as to administer an amount of the active compound(s) that is effective to achieve the desired therapeutic response for a particular subject. The selected dosage level will depend upon the activity of the therapeutic composition, the route of administration, combination with other drugs or treatments, the severity of the condition being treated, and the condition and prior medical history of the subject being treated. However, it is within the skill of the art to start doses of the compound at levels lower than required to achieve the desired therapeutic effect and to gradually increase the dosage until the desired effect is achieved. The potency of a composition can vary, and therefore a “treatment effective amount” can vary. However, using the assay methods described herein, one skilled in the art can readily assess the potency and efficacy of a candidate compound of the presently disclosed subject matter and adjust the therapeutic regimen accordingly.

After review of the disclosure of the presently disclosed subject matter presented herein, one of ordinary skill in the art can tailor the dosages to an individual subject, taking into account the particular formulation, method of administration to be used with the composition, and particular disease treated. Further calculations of dose can consider subject height and weight, severity and stage of symptoms, and the presence of additional deleterious physical conditions. Such adjustments or variations, as well as evaluation of when and how to make such adjustments or variations, are well known to those of ordinary skill in the art of medicine.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments.

All documents mentioned herein are incorporated herein by reference. All publications and patent documents cited in this application are incorporated by reference for all purposes to the same extent as if each individual publication or patent document were so individually denoted. By their citation of various references in this document, Applicants do not admit any particular reference is “prior art” to their invention. Embodiments of inventive compositions and methods are illustrated in the following examples.

### EXAMPLES

The following non-limiting Examples serve to illustrate selected embodiments of the invention. It will be appreciated that variations in proportions and alternatives in elements of the components shown will be apparent to those skilled in the art and are within the scope of embodiments of the present invention.

### Example 1

### TNFRSF25 Modulates T Regulatory Cells, TH17 and TH2 Polarization

Materials and Methods

Cloning and characterization of mouse and human TNFR25 isoforms: Messenger RNA was extracted from resting and concanavalin A (5 μg/ml) activated mouse splenocytes with the Micro Fast-Track kit (Invitrogen, Carlsbad, Calif.) and was converted to cDNA using the Superscript II kit from the same manufacturer. RT-PCR was performed using primers to TNFR25 exons 2-7, 4-10 and 1-10. The products were resolved on an agarose gel and subcloned into the PCR II vector using the TOPO cloning kit (Invitrogen) and were confirmed as splice forms of mTNFR25 by sequencing.

Activation induced alternative splicing of TNFR25 (DR3) was studied with human cells because splicing products could be better separated after PCR by agarose gel electrophoresis. Human peripheral blood mononuclear cells (PBMCs) were isolated from healthy donors by Ficoll-Hypaque density gradient centrifugation. 5×10⁶ cells per sample were activated with PHA (5 μg/ml), or immobilized anti-hCD3 (OKT3, 5 μg/ml) and anti-hCD28 (1 μg/ml), or PMA (10 ng/ml) and ionomycin (400 ng/ml). To block protein synthesis or protein kinase C, cells were pretreated with cycloheximide (10 μg/ml) or H7 (50 μM) respectively for 30 min prior to activation. The cells were harvested at the indicated time points and mRNA extracted and converted to cDNA as described above. Human β-actin was used as internal control. Quantification of PCR products was done with Molecular Analyst software (Bio-Rad, Hercules, Calif.).

Animals: Wild type C57BL/6 and Balb/c mice were purchased from Charles River Laboratories (Wilmington, Mass.). TCR transgenic OT-I mice (University of Washington, Seattle, Wash.). Full length TNFR25 (FL-TNFR25), splice variant lacking exons 5-6 (Δ5,6-TNFR25) and dominant negative TNFR25 lacking the intracellular domain (DN-TNFR25) transgenic mice were produced by the UM transgenic animal facility. The corresponding DNA fragments were cloned into human CD2 promoter and enhancer vector. Expression cassettes were excised with Not I digestion and after purification were microinjected into fertilized eggs. Potential founders were screened by PCR from tail biopsies and were bred into C57BL/6 and BALB/c backgrounds. Mice were used at 6-12 weeks of age and were maintained in pathogen-free conditions at the UM Animal facilities. All animal use procedures were approved by the University of Miami Animal Care and Use Committee.

Antibodies and fusion proteins: Hybridomas producing signaling antibodies to mouse CD3 (2C11) and to mouse OX40 (OX86) were obtained from ATCC (Manassas, Va.) and HPACC (Salisbury, UK) respectively. Armenian hamster hybridomas producing antibodies to mouse TNFR25 (4C12, agonistic) and TL1A (L4G6, blocking) were generated as described previously (Fang, L., B. Adkins, V. Deyev, and E. R. Podack. 2008. J. Exp Med 205:1037-1048). Antibodies were produced in hollow fiber bioreactors (Fibercell Systems, Frederick, Md.) and purified from serum-free supernatants on a protein G column (GE Healthcare, UK). Signaling antibodies to mouse CD28 and mouse GITR (DTA-1) and mouse Foxp3 staining kit were purchased from eBioscience (San Diego, Calif.).

The DNA fragment encoding soluble mTL1A was cloned in the pMal-c2x vector (NE Biolabs, Ipswich, Mass.) in frame with malE gene encoding maltose binding protein (MBP) and a fragment encoding the neck region of human surfactant D. The resulting MBP-neck-TL1A fusion protein was produced in the Rosetta-gami E. coli strain from Novagen (EMD Biosciences, Gibbstown, N.J.) and purified from bacterial lysate on an amylose column (NE Biolabs).

Purification of T cell subsets: Mouse CD4 or CD8 T cells were purified from spleens, thymuses or inguinal lymph nodes by negative selection (SpinSep kit by Stemcell Technologies, Vancouver, BC) according to the manufacturer's protocol. Wild type or transgenic CD4⁺CD25⁺ and CD4⁺CD25⁻ cells were purified from mouse splenocytes using Miltenyi Biotec (Auburn, Calif.) Treg isolation kit. OT-I cells were magnetically isolated as CD8⁺ fraction from spleen cells of OT-I TCR transgenic mice using CD8a-specific beads from the same manufacturer. The purity was routinely around 92%-98% as examined by flow cytometry.

Flow cytometry analysis: Single cell suspensions were prepared from thymus, spleen, or inguinal lymph nodes. 10⁵ cells were pre-blocked with anti-mouse CD16/CD32 and stained with CD4-FITC, CD8-Cyc (BD Pharmingen, San Jose, Calif.), and Armenian hamster anti-mouse TNFR25 or control hamster IgG for 30 minutes at 4° C. Cells were washed in FACS buffer (PBS containing 0.5% BSA and 2 mM EDTA) and then treated with human IgG for 5 min at 4° C., followed by staining with goat anti-Armenian hamster IgG-Biotin (Jackson Immunoresearch, West Grove, Pa.)) for 30 minutes at 4° C. Cells were washed in FACS buffer and then stained with Streptavidin-PE or Streptavidin-Cychrome (BD Pharmigen) for 30 minutes at 4° C. Samples were analyzed using a Becton Dickinson FACS LSR instrument and CELLQuest software. B220, NK1.1 and CD11c FITC and PE labeled antibodies were similarly combined with anti-mouse TNFR25 staining to detect TNFR25 expression levels in corresponding cell populations.

Cytokine ELISA: CD4 cells were isolated from the spleens of wild type and transgenic mice and activated with pate bound anti-CD3 (2 μg/ml) and soluble anti-CD28 (1 μg/ml). Supernatants of primary cultures were collected on day 3.

For polarization experiments, CD4 cells were activated with anti-CD3 and anti-CD28 alone (ThN), or combined with IL-12 (5 ng/ml) and anti-IL-4 (20 μg/ml) for Th1 polarization; or with IL-4 (10 ng/ml), anti-IFN-γ (10 μg/ml) and anti-IL-12 (10 μg/ml) for Th2. To induce Th17, the cells were activated in the presence of IL-1β (10 ng/ml), TGF-β (5 ng/ml) and IL-6 (20 ng/ml) with 10 μg/ml of anti-IFN-γ and 5 μg/ml of anti-IL-4. On day 4, cells were washed and re-plated with anti-CD3 for 24 h, after that the supernatants were collected and analyzed by ELISA.

Antibody pairs and standards from BD were used for IL-2, IFN-γ, IL-4 and IL-17 analysis. Reagents for IL-13 ELISA were purchased from R&D Systems (Minneapolis, Minn.) and reagents for IL-5 and IL-10 ELISA were purchased from eBioscience. The assays were performed according to manufacturer's protocols.

T cell proliferation assay: Splenocytes or purified CD4 and CD8 lymphocytes were plated in triplicate at 1×10⁵ cells/well in 96-well flat-bottomed plates. Cells were activated with immobilized anti-CD3 (2 μg/ml) with or without soluble anti-CD28 (1 μg/ml), or concanavalin A (5 μg/ml), or PMA (10 ng/ml) with ionomycin (400 ng/ml). Recombinant mouse IL-2 was added to the cultures at 1000 U/ml in indicated experiments. Cells were cultured for 72 h and pulsed for the last 6 h of incubation with 1 μCi/well of 3H-thymidine (Perkin Elmer, Waltham, Mass.). Incorporated isotope was measured by liquid scintillation counting (Micro Beta TriLux counter, Perkin Elmer).

In vitro assay for Treg function: 1×10⁵ of CD4⁺CD25⁻ cells were placed in 96-well plates and activated with soluble anti-CD3 (2C11) antibody 1 μg/ml in the presence or absence of titrating numbers of CD4⁺CD25⁺ regulatory cells. Control IgG, 4C12, OX86 and DTA-1 antibodies or recombinant MBP-TL1A fusion protein were added to some wells at indicated concentrations.

For OT-I proliferation assay, purified CD8 lymphocytes from OT-I mice were stimulated with titrating concentrations of SIINFEKL (SEQ ID NO: 1) peptide (UM protein sequencing facility) in the presence or absence of Tregs and antibodies, or with 10⁻¹¹ M SIINFEKL (SEQ ID NO: 1) in the presence of titrating numbers of CD4⁺CD25⁺ lymphocytes.

Cultures were incubated for 72 h and pulsed with ³H-thymidine for the last 4-6 h of incubation.

Results:

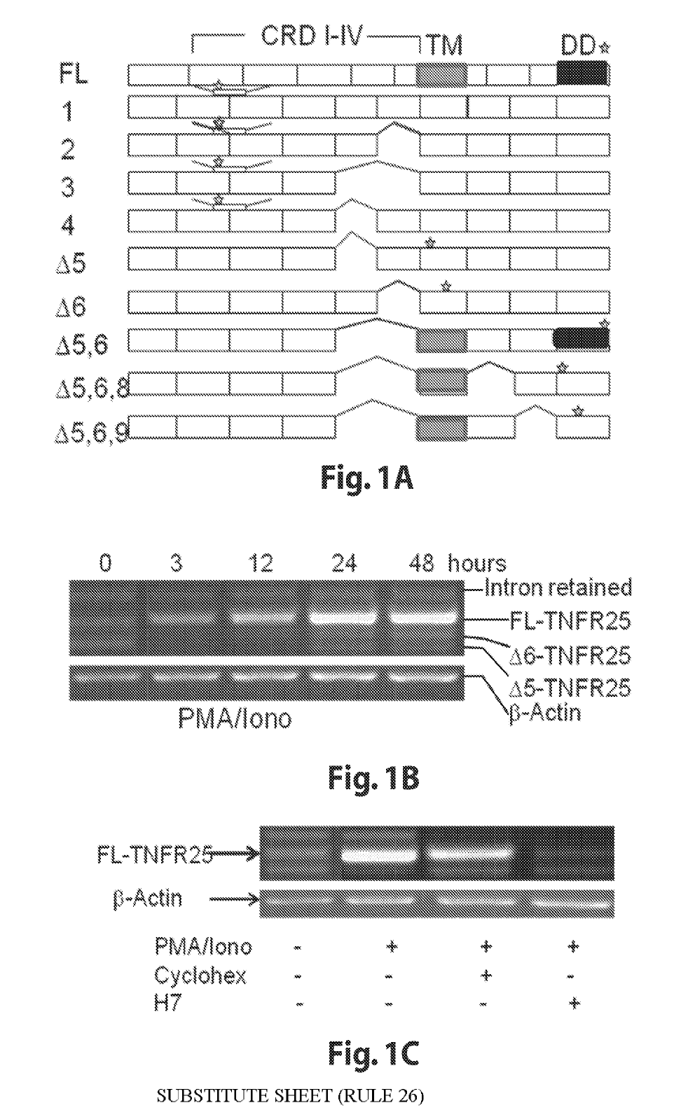

Protein kinase c inhibitor blocks splicing of murine TNFR25 mRNA in activated lymphocytes: Eleven randomly spliced TNFR25 transcripts, named LARD, were described in human lymphocytes (Screaton, G. R., et al. 1997. Proc Natl Acad Sci USA 94:4615-4619). Analyzing murine TNFR25 transcripts, constitutive transcription and random splicing of TNFR25 was found to be conserved between mouse and man. Ten murine splice variants, including the full length (FL) transcript and transcripts that had retained an intron were identified by sequencing (FIG. 1A). Transcripts 1-4 retaining an intron are not likely to form functional proteins upon translation due to the introduction of an early translation termination signal. The splice forms designated Δ5 and Δ6, lacking exons 5 or 6 respectively not containing a transmembrane domain are most likely secreted and, if capable of binding to the ligand, could act as TL1A inhibitors. The splice form designated as Δ5, 6-TNFR25 lacks both exons 5 and 6 but retains the reading frame; it encodes a transmembrane protein with complete death domain, but lacking the fourth cysteine-rich domain in its extracellular part. When transfected into murine tumor cells P815 or EL4 and triggered with soluble TL IA or with an agonistic antibody 4C12, this receptor isoform signaled caspase cleavage and cell death similarly to the FL-TNFR25 isoform; however ligand-binding properties of the Δ5, 6 isoform may differ due to the deletion of cystein-rich domains in the extracellular region. The next two splice forms are also membrane associated, however Δ5,6,8 and Δ5,6,9 lack the cytoplasmic death domain indicating impaired signaling function.

Splicing of murine or human TNFR25 to the full length (FL) transcript is initiated trough lymphocyte activation by anti-CD3, PHA, PMA alone or in combination with Ca ionophores. Because human TNFR25 splice forms are more easily resolved by electrophoresis than murine TNFR25, splicing was studied in human PBL. (FIG. 1B). Full length TNFR25 is readily detectable by PCR within 3 h after activation and is associated with a decrease in randomly spliced forms suggesting alteration of splicing rather than transcriptional regulation. At later time points transcriptional activation may also contribute to upregulation of spliced TNFR25 transcripts, as suggested by the intensity of the TNFR25-band after 24 and 48 hours (FIG. 1B). Splicing is not inhibited by cycloheximide and therefore is not dependent on new protein synthesis. However H7, an inhibitor of protein kinase C (FIG. 1C), completely blocked the generation of FL-TNFR25, consistent with the ability of PMA to activate protein kinase C and initiate splicing of TNFR25 (FIG. 1B). As shown previously, T cell activation results in upregulation of TNFR25 protein expression detectable by flow cytometry (Fang, L., B. et al., 2008. J Exp Med 205:1037-1048). Human and mouse splicing requirements for TNFR25 are similar.

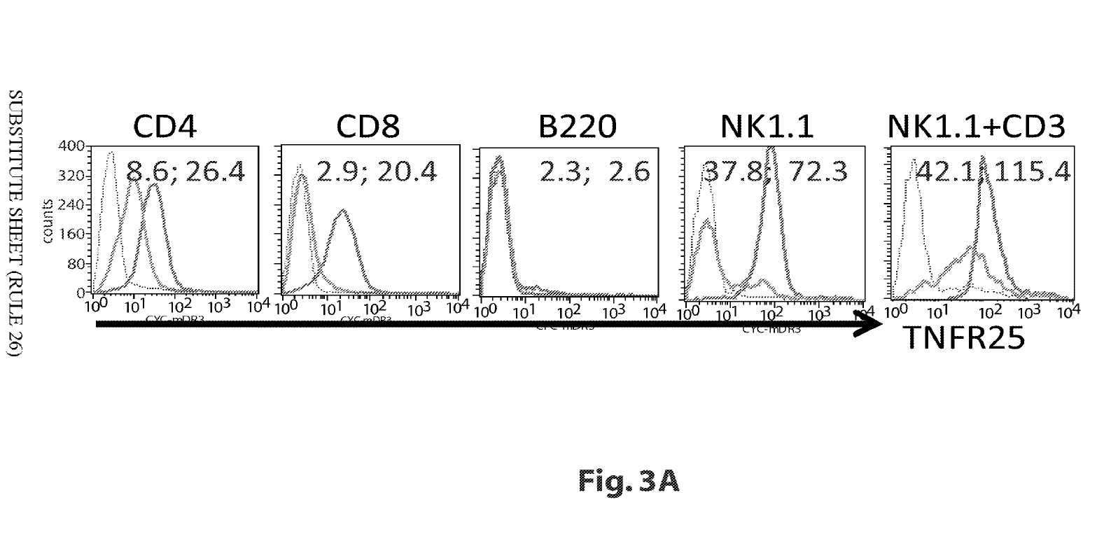

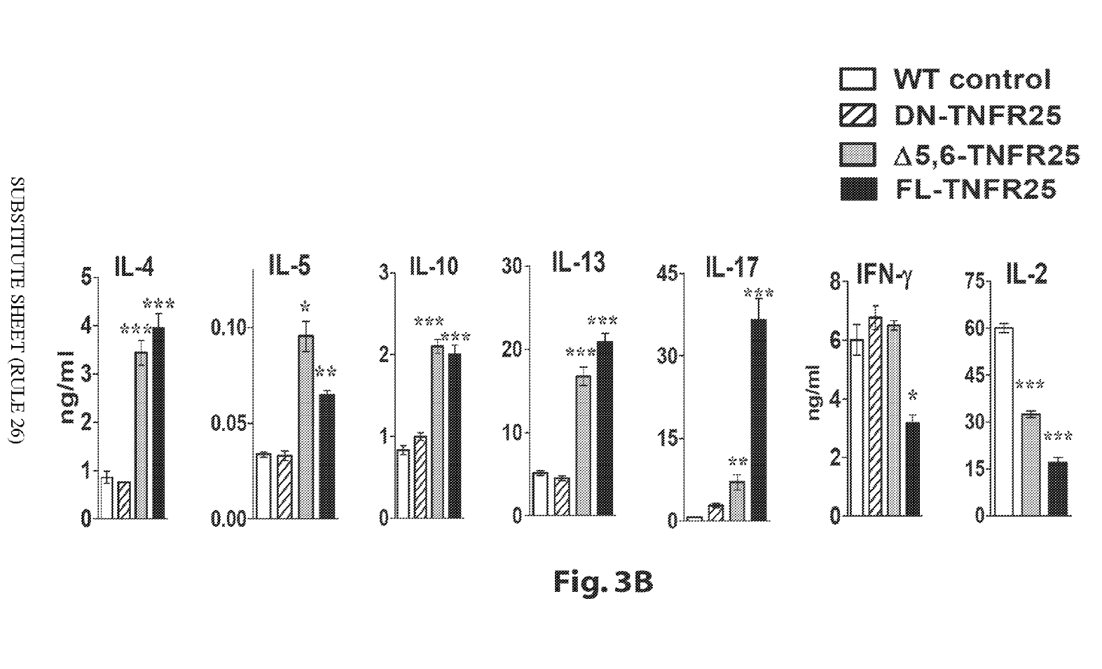

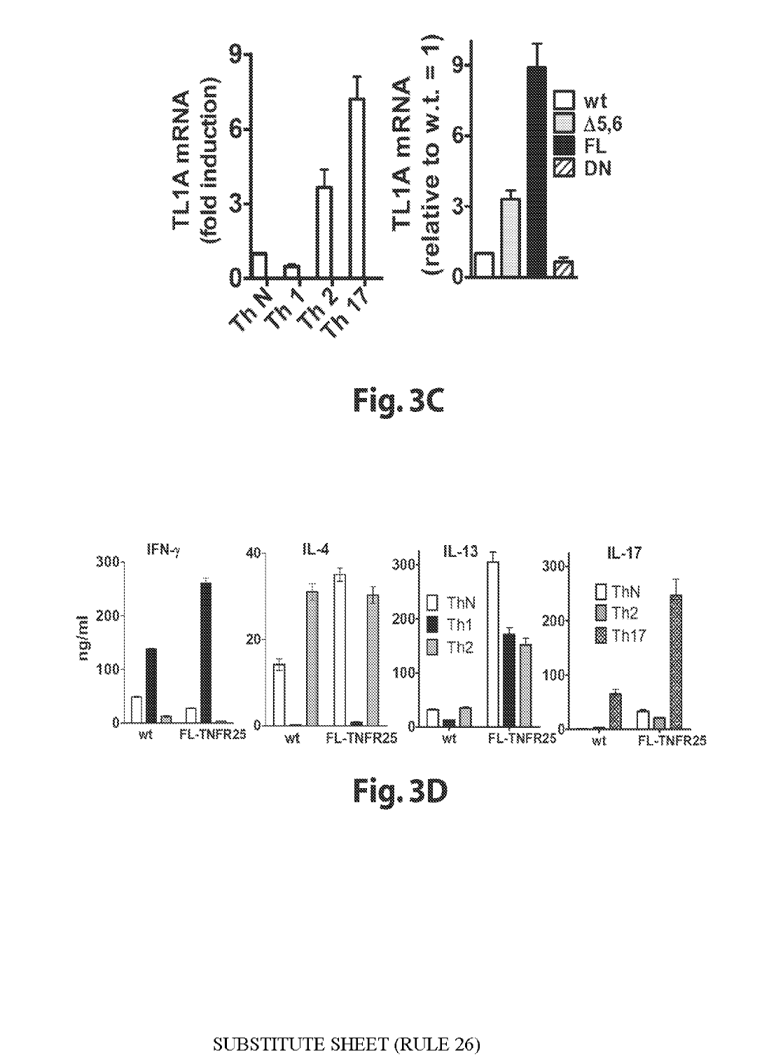

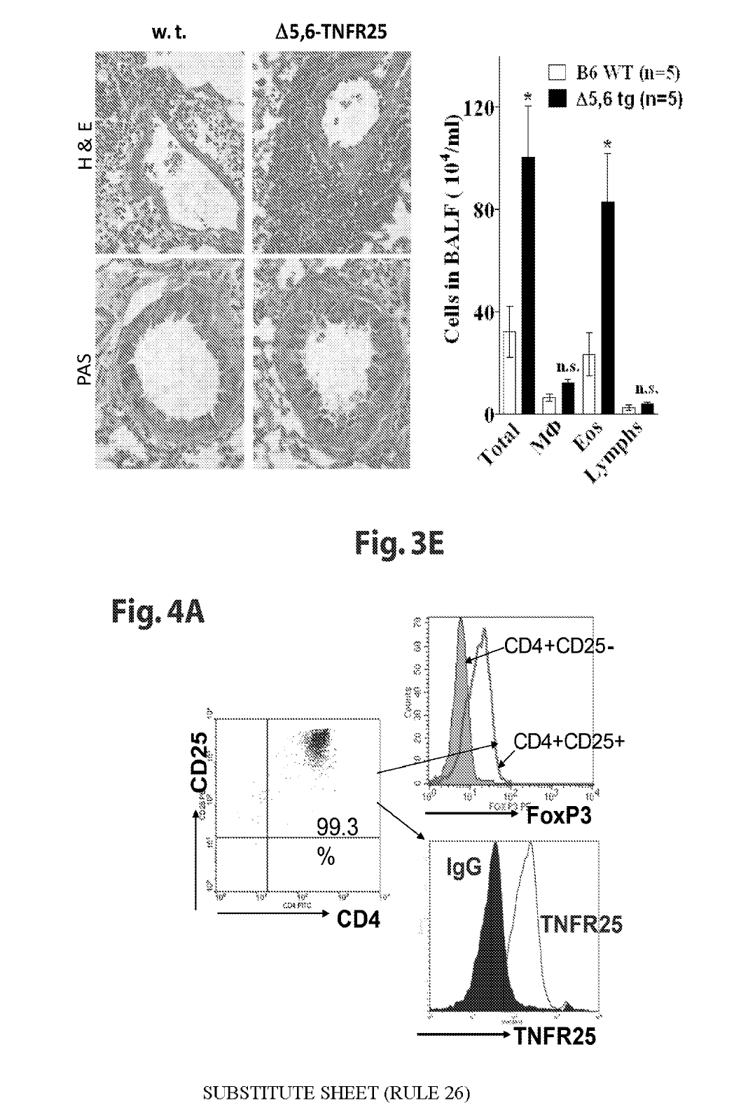

Transgenic expression of TNFR25 results in decreased T cell numbers and proliferation defect: Three types of transgenic mice were generated, expressing FL-TNFR25, the □5,6-TNFR25 splice a dominant negative (DN) mutant of TNFR25 under the CD2 promoter and locus control region. High level expression of FL-TNFR25 and Δ5,6-TNFR25 was found in all T cell subpopulations including NKT cells and in NK cells, while B cells were transgene negative (FIG. 3A).

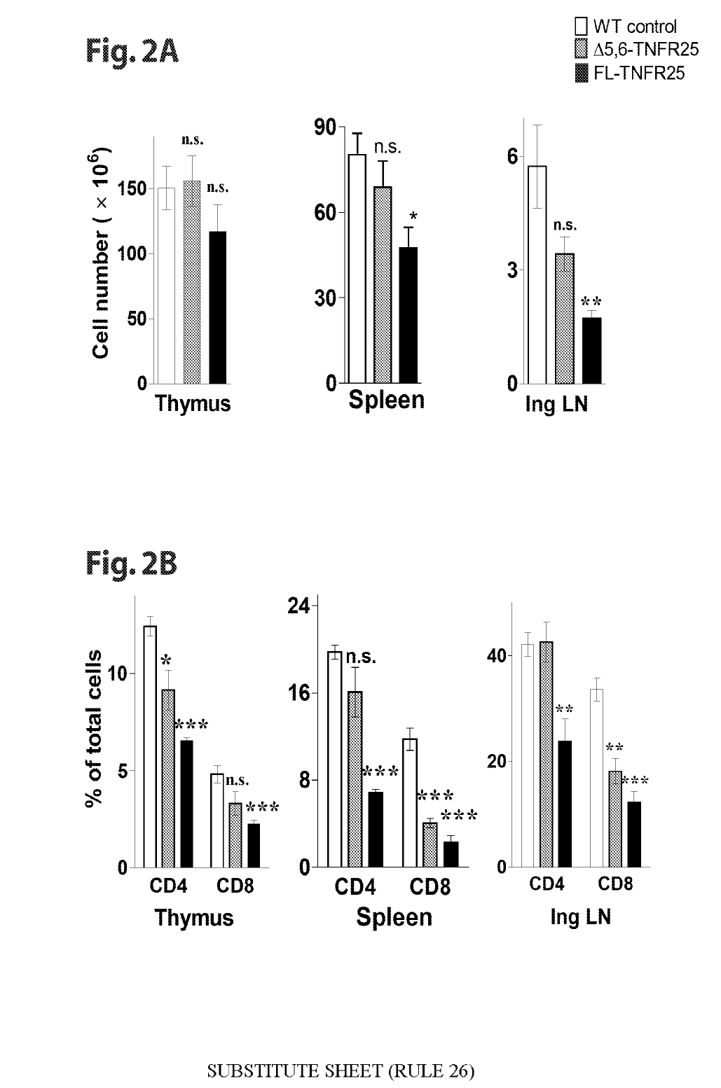

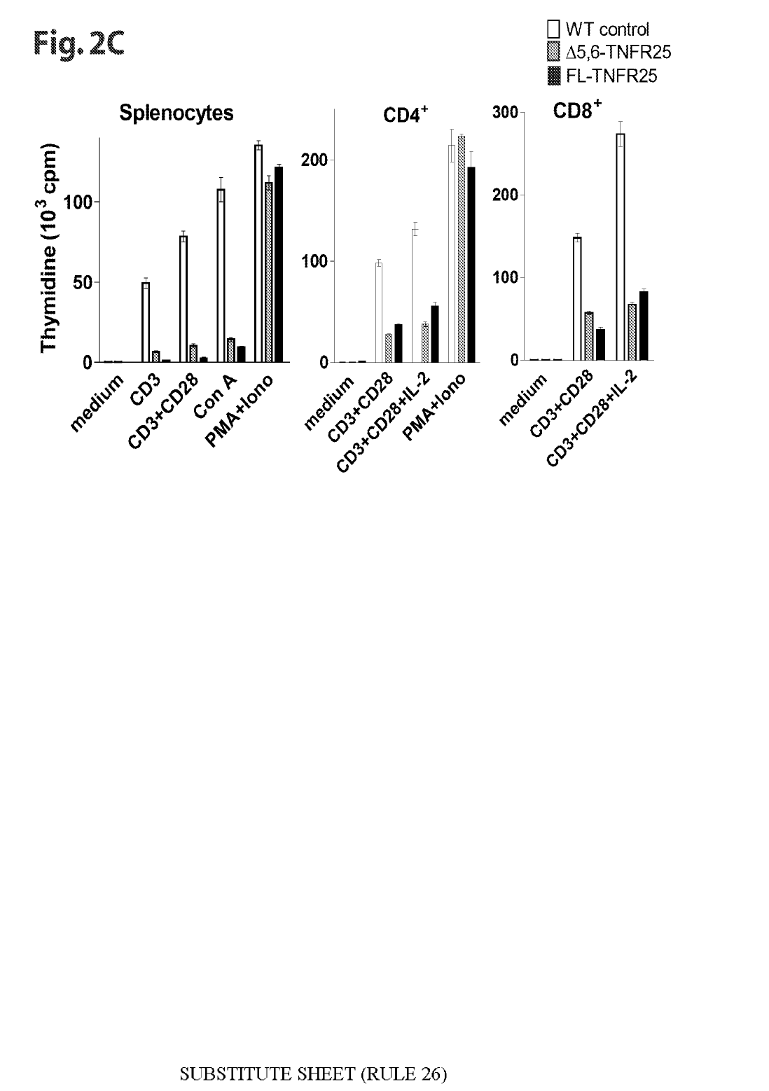

Transgenic mice thrived normally and did not exhibit any pathological signs. However upon immunological analysis several abnormalities were noted, suggesting that increased constitutive signaling or signaling induced by TL IA ligation of transgenic TNFR25 is occurring. Transgenic overexpression of FL-TNFR25 and Δ5,6-TNFR25, but not DN-TNFR25, caused moderately diminished cellularity of the spleen and lymph nodes (FIG. 2A) with a significant decrease in the numbers of CD4 and CD8 cells in thymus, spleen and lymph nodes (FIG. 2B). The CD4 to CD8 ratio in transgenic mice was not affected. DN-TNFR25 transgenic mice, in which TNFR25 signals are blocked due to the over expression of the dominant negative transgene, had normal cell numbers, indicating that TNFR25 signaling is not required and not normally occurring for the formation of the normal T cell repertoire in w.t. mice. The decrease of T cell numbers in FL- and Δ5,6-TNFR25 transgenic mice was associated with a diminished ability of TNFR25 transgenic CD4 and CD8 T cells to proliferate in response to TCR signals (FIG. 2C), even when costimulated by CD28 or IL-2 was added. DN-TNFR25 transgenic T cells, in contrast, proliferated normally, indicating that increased signals by generated by transgenic TNFR25 are responsible. Both FL- and Δ5,6-TNFR25 transgenic T cells were able to proliferate normally in response to PMA and ionomycin (FIG. 2C), indicating effects of TNFR25 signaling upstream but not downstream of PKC. The diminished cellularity and rate of proliferation of T cells obtained from FL- and Δ5,6-TNFR25 transgenic mice could not be explained by an increased rate of apoptosis as determined by annexin-V staining and AAD uptake three days after activation. TNFR25 triggering with an agonistic antibody did not induce caspase cleavage and cell death in transgenic T lymphocytes unlike TNFR25 overxpressed in transfected EL4 and P815 cells. Transgenic T cells after activation upregulated CD25 to the same extent as cells from wild type litter mates. However TNFR25 transgenic T cells produced significantly less IL-2 after 72 h of TCR stimulation in combination with anti-CD28, but IL-2 addition did not restore proliferation as mentioned above (FIGS. 2C and 3B).

FL-TNFR25 and Δ5,6-TNFR25 have the identical cytoplasmic signaling domain and differ in the extracellular domain. Their level of transgenic expression is similar. However the effects on cellularity and proliferation were more pronounced in FL-TNFR25 transgenic cells, suggesting stronger signaling potentially due to higher affinity interaction with the natural TL1A ligand. Chronic signaling by TNFR25 occurring in transgenic mice apparently downregulates sensitivity to TCR signals upstream of PKC, a process superficially similar to desensitization of other receptors. The precise mechanism requires further studies.

TNFR25 transgenes induce increased production of Th2 and Th17 cytokines in activated CD4 cells: Analysis of cytokine secretion by transgenic CD4 cells revealed that FL- and Δ5,6-TNFR25 transgenic cells secrete high levels of the Th2 cytokines IL-4, IL-5, IL-10, IL-13 and IL-17 upon primary activation, when compared to wild type or DN-TNFR25 transgenic cells (FIG. 3B). IFN-γ secretion on the other hand was not increased while IL-2 was diminished. The finding that IL-4 secreted by FL- and Δ5,6-TNFR25 was elevated already 24 h after activation, indicates that the FL- and Δ5,6- but not DN-TNFR25 transgenic cells are spontaneously biased to Th2 polarization. Importantly, despite their Th2 bias, TNFR25 transgenic cells can be polarized to Th1 in the presence of IL-12 and IFN-γ and blocking antibody to IL-4. Strikingly, Th1 polarized FL-TNFR25 transgenic CD4 cells produce higher levels of IFN-γ than wild type (w.t). TH1 polarized cells, but surprisingly also, IL-13 (FIG. 3D). These data support the finding that TNFR25 signaling is of particular importance for IL-13 production under Th1 polarizing conditions and can costimulate IFN-γ. The TH2 bias of TNFR25 transgenic mice and increased IL-13 production suggested increased susceptibility to allergic lung inflammation underlying atopic asthma. Comparing wild type with FL- and Δ5,6-TNFR25 transgenic mice in the ovalbumin model of allergic asthma, a strong increase in lung inflammation was confirmed in the transgenic mice (FIG. 3E). In contrast, DN-TNFR25 transgenic mice, whose CD4 cells are resistant to Th2 polarization, are also resistant to lung inflammation in the ovalbumin model.

The strongest effect of TNFR25 transgenes in activated CD4 cells is on IL-17 production, which is significantly increased upon primary stimulation of FL-TNFR25 and to a lesser extent of Δ5,6 TNFR25 transgenic CD4 cells (FIG. 3B). Increased IL-17 production is also observed after secondary stimulation of TH17 polarized TNFR25 transgenic cells (FIG. 3D), indicating that TNFR25 signals are costimulatory for IL-17 production and for Th2 cytokine production as shown in the murine asthma model. TH17 and to a lesser extent TH2 polarization upregulate the expression of TL1A on CD4 cells (FIG. 3C). TNFR25 polarization in contrast does not affect TNFR25 expression.

TNFR2525 signals suppress Treg-mediated inhibition of CD4 and CD8 effector cell proliferation: Resting CD4 cells containing T regulatory (Treg) cells express low levels of TNFR25 (FIG. 2A), which is upregulated upon activation (FIG. 1) or in transgenic mice (FIG. 2A). Purified and cultured CD4⁺ CD25⁺ FoxP3⁺ Treg that have been expanded with anti CD3/anti CD28 beads in the presence of high IL-2 also express high levels of TNFR25, raising the question of the function of TNFR25 in Treg. In FL- and Δ5,6- but DN-TNFR25-transgenic mice the absolute number of CD4 cells is reduced, however, the frequency of FoxP3+CD4 cells is increased (Table 1) indicating that TNFR25 signaling in transgenic mice favors the development of Treg. This increase of FoxP3⁺CD4 cells is due to an increase in the CD25 negative, FoxP3⁺ population.

TABLE 1
Treg frequencies in spleen cells of wild type and transgenic mice.
Single cell suspensions of wild type and transgenic splenocytes
were stained for CD4, CD25 and Foxp3 and analyzed by
flow cytometry.

| | | | | Foxp3+ |
|---|---|---|---|---|
| | CD4+ in spleen | Foxp3+ | Foxp3+CD25+ | CD25− |
| | % of total | % of CD4 | % of CD4 | % of CD4 |
| Wild type | 30.3 | 13.5 | 10.9 | 2.6 |
| Δ5,6-TNFR25 | 17.9 | 14.4 | 9.8 | 4.6 |
| FL-TNFR25 | 11.0 | 23.9 | 11.7 | 12.2 |
| DN-TNFR25 | 30.7 | 12.8 | 11.1 | 1.7 |

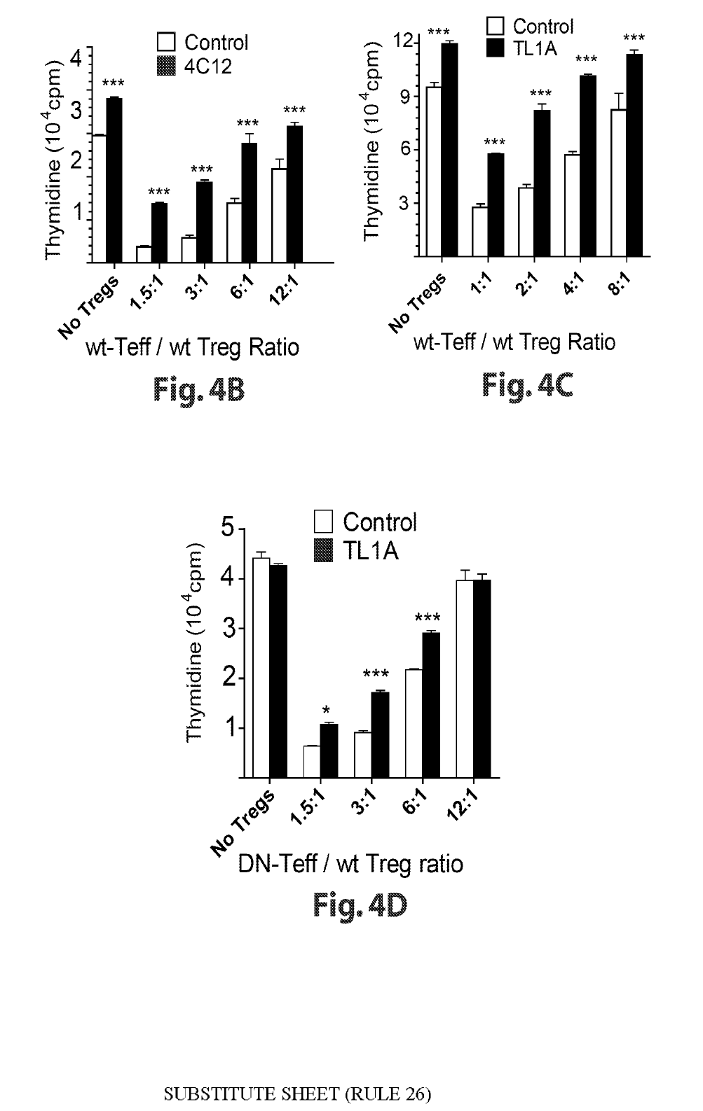

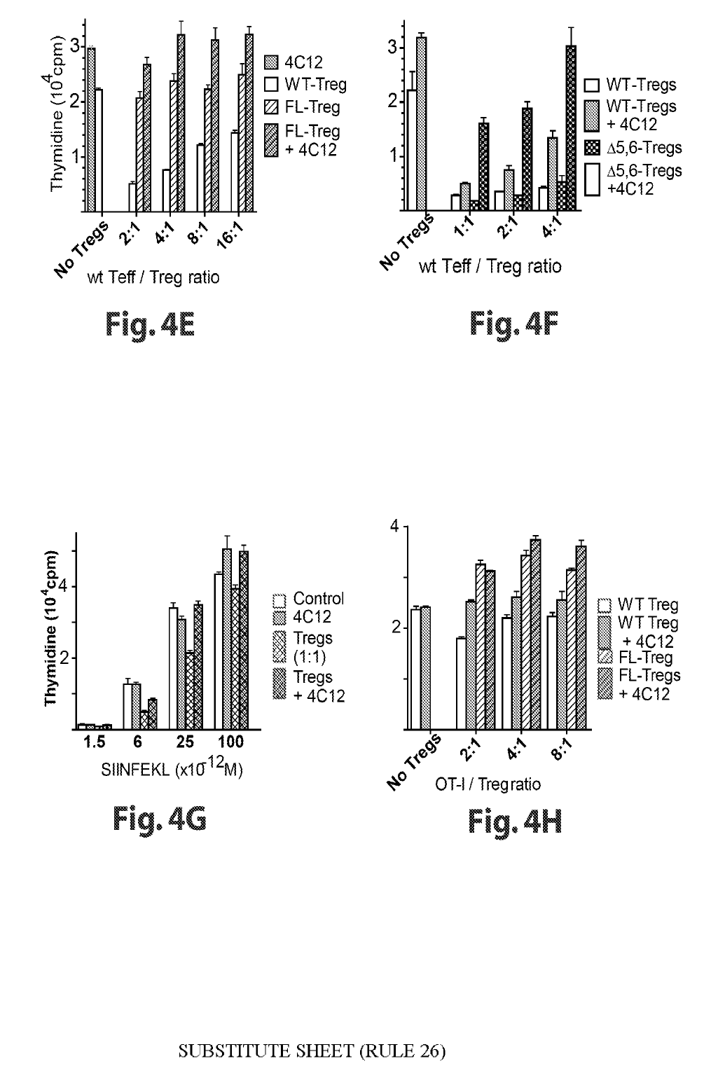

One interest was to determine the effect of TNFR25 signaling on Treg suppressive activity. TNFR25 signaling was achieved with the agonistic TNFR25 antibody 4C12, characterized previously (Fang, L., B. et al., 2008. J Exp Med 205:1037-1048), and with soluble TL1A. Both, the agonistic TNFR25 antibody 4C12 and TL1A costimulated proliferation of non-transgenic (wt) purified CD4+CD25− Teff (FIG. 4A). Unlike chronic overexpression of TNFR25 which inhibits proliferation (FIG. 2), acute signaling in activated effector cells is costimulatory. Adding Treg caused dose dependent inhibition of Teff proliferation; this suppressive effect of Treg on proliferation was inhibited by the agonistic TNFR25 antibody 4C12 or by soluble TL1A (FIGS. 4A, B). CD4^{+CD}25⁺ Treg cells alone displayed minimal proliferation upon stimulation with anti-CD3 even in the presence of TNFR25 agonists. To confirm the suppressive effect of TNFR25 signals on Treg in the proliferation assay, DN-TNFR25-transgenic CD4 effectors were used, which are unable to respond to TNFR25 agonists due to the presence of the dominant negative TNFR25 transgene which suppresses signaling by endogenous TNFR25. The results showed that DN-TNFR25 Teff are resistant to costimulation by TL1A and do not respond with increased proliferation (FIG. 4C). However, TL1A is still able to provide relief from inhibition by Treg indicating that TL inhibits the suppressive activity of Treg.

FL-TNFR25 Treg are not inhibitory in the proliferation assay, even in the absence of added TL1A or agonistic antibody 4C12 (FIG. 4D), indicating that chronic TNFR25 signaling abolishes Treg activity in this assay. Δ5,6-TNFR25 transgenic Treg, in contrast, retain inhibitory activity, but are more susceptible to the counter-suppressive effect of 4C12 than wild type Treg (FIG. 4E). This difference in inhibitory capacity of FL and Δ5,6 transgenic Treg indicates that relatively subtle differences in ligand binding affinity of TNFR25, controlled by alternative splicing, can have major functional effects.

Unlike CD4 cells, CD8 T cells are not costimulated by the agonistic TNFR25 antibody 4C12. CD4⁺CD25⁺ Treg inhibit proliferation of TCR transgenic CD8 effector cells (OT-I) stimulated by the cognate peptide and this inhibition is relieved by 4C12 (FIG. 4F). As in CD4 proliferation assays, FL-TNFR25 transgenic Treg had no inhibitory activity for CD8 proliferation (FIG. 4G).

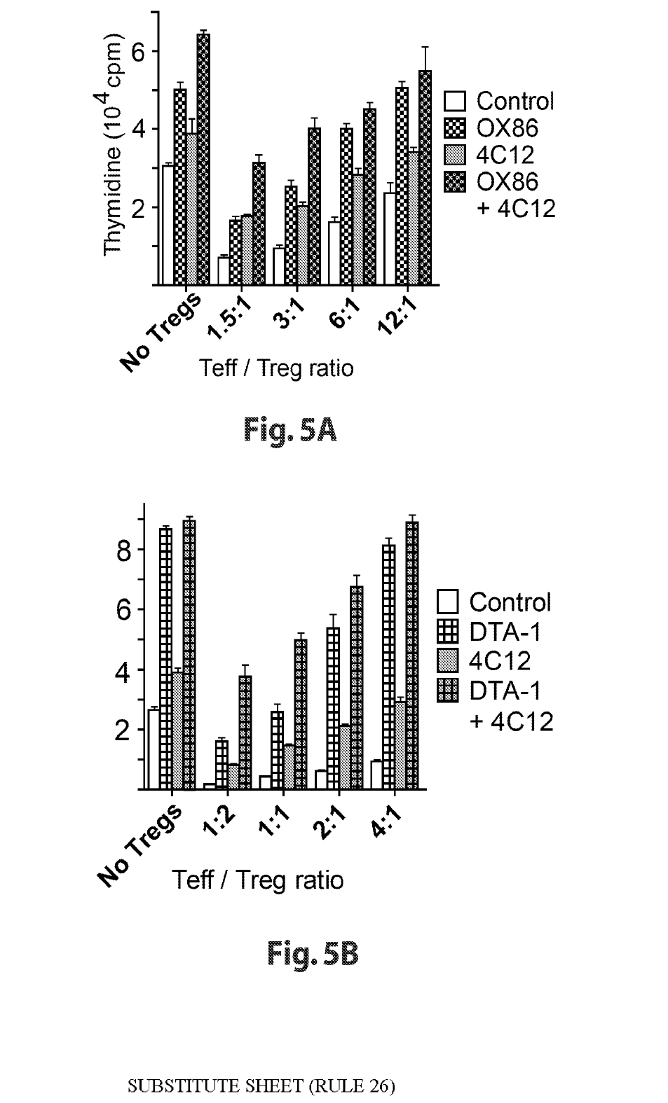

TNFR25 compared to OX40 and GITR is primarily an inhibitor for Treg and only modest costimulator for Teff The TNF receptor family members OX40 (CD134, TNFRSF4) and GITR (TNFRSFI8) costimulate effector cells and inhibit Treg (Shimizu, J., S. et al., 2002. Nat Immunol 3:135-142; Tone, M., et al. 2003. Proc Natl Acad Sci USA 100:15059-15064; Valzasina, B., et al. 2005. Blood 105:2845-2851; Vu, M. D., et al. 2007. Blood 110:2501-2510). In order to understand the role of these three receptors in T cell function, their costimulatory activity on Teff and inhibitory activity on Treg was compared with the help of agonistic antibodies. The OX40 agonistic antibody OX86 showed stronger costimulatory activity for CD4 effector cells as compared to the TNFR25 agonist 4C12. However, OX86 and 4C12 had comparable effects on proliferation in the presence of high Treg numbers. At lower Treg numbers, the costimulatory effect of OX86 on Teff overwhelms its effect on Treg inhibition (FIG. 5A). The agonistic GITR antibody DTA-1 had even stronger costimulatory activity for CD4 effectors than 4C12 or OX86 and could overcome Treg inhibitory activity even at high Treg numbers (FIG. 5B).

The data indicate that OX40 and GITR activity is focused primarily on costimulating Teff, making the resistant to inhibition by Treg. In contrast, TNFR25 primarily acts on Treg with relatively modest activity on T effector cells.

Discussion:

These studies identify TNFR25 as a novel, heretofore unrecognized regulator of CD4⁺ Treg cells. TNFR25 signals attenuate the suppressive activity of regulatory cells without costimulating CD8 T effector cells and only modestly costimulating CD4 T effector cells. These properties distinguish TNFR25 from OX40 and GITR, which have strong costimulatory effects on T effector cells while simultaneously inhibiting Treg. Another distinguishing feature of TNFR25 is its control of expression by mRNA splicing upon activation of PKC. In this manner TNFR25 is expressed within hours in TCR activated cells ready to engage the ligand and mediate TNFR25 signals. The nature/strength of the signals delivered by TNFR25 depends on its composition, which is determined by the TNFR25 splice form. Δ5,6-TNFR25 lacking two exons in the extracellular domain was studied in more detail in transgenic mice. Compared to FL-TNFR25, the effects of the Δ5,6-form on cell number, cytokine expression and on regulation of Treg was diminished. FL-TNFR25 transgenic Treg had no regulatory activity in the proliferation assay, while Δ5,6-TNFR25 transgenic Treg were fully active, but highly susceptible to TNFR25 agonists due to the high level of transgene expression. Similarly, FL-TNFR25 but not Δ5,6 expression induced a large increase in CD25⁻, FoxP3⁺CD4 cells. Alternative splicing, probably regulated by factors in the local environment, therefore can modulate the functional effects of TNFR25 depending on local needs. Several splice forms of TNFR25 are soluble and others lack part of the intracellular domain, which if expressed can further fine tune ligand binding or signaling, respectively. How important alternative splicing is in the regulation of pathophysiological immune responses remains to be established.

Functional differences observed in FL- and Δ5,6-TNFR25 transgenic mice compared to wild type mice indicates increased signaling by the transgenic receptors either by constitutive signaling or by interaction with homeostatically expressed TL1A. The functional differences between FL- and Δ5,6-transgenic mice suggest the latter hypothesis, since Δ5,6-TNFR25 due to the lack of two exons, while still binding TL1A, is likely to have lower affinity and therefore attenuated effects. This is reflected in vivo by a less drastic reduction of cell numbers and proliferation in Δ5,6-TNFR25 transgenic compared to FL-TNFR25 transgenic mice. Mechanistically TNFR25 signals may involve non-receptor tyrosine protein kinases, which, as shown for Itk, can be involved not only in the regulation of T cell proliferation, but in Th1/Th2 differentiation as well. In contrast, blocking of TNFR25 with a dominant negative mutant had little effect on T cell proliferation and primary cytokine production upon TCR stimulation.

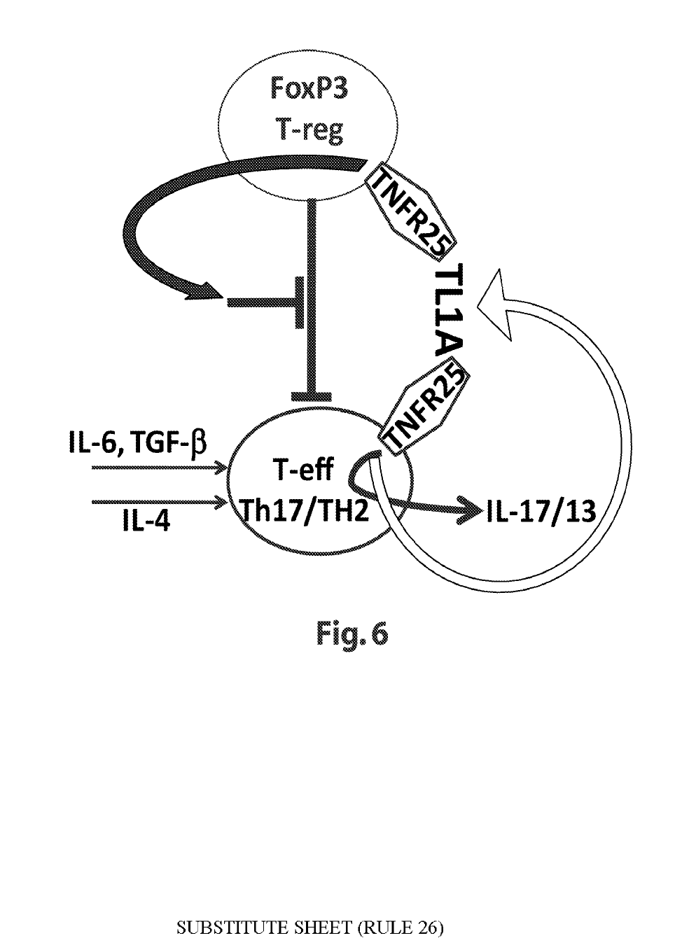

In addition to effects on TH2 polarization, TNFR25 has strong effects on IL-17 production and on TL1 A expression by CD4 cell. This places TNFR25 into a central position for the regulation of inflammatory responses, which may be of particular importance in the mucosal compartment. Conditions favorable for TH17 polarization and IL-17 production result in the upregulation of TL1A expression by CD4 cells. TL IA binding to TNFR25 mediates additional IL-17 production thereby maintaining the inflammatory response. Similar to TNF-α, TL1A is rapidly released from cells and free to diffuse into the surrounding area, where TL1A can suppress the activity of Treg thereby further supporting inflammation (FIG. 6). Transgenic TNFR25 causes significant increases in the frequency of FoxP3⁺CD4 cells. This function is different from TNF which decreases FoxP3 expression while also down-modulating Treg. However, the increased TNFR25 expression and signaling abrogated the inhibitory activity in the proliferation assay and increased IL-17 production and TH17 polarization. TNFR25 thus has a dual role in regulating inflammatory cytokine production and modulating Treg activity including the frequency of FoxP3 positive cells. More research is needed to understand the balance of apparently opposing effects by TNFR25 in the tuning of inflammatory responses.

The role of TL1A and TNFR25 in regulating inflammatory responses and Treg activity is consistent with increased TL1A expression observed in inflammatory bowel disease (IBD, Morbus Crohn), which is now recognized as TH17 disease. The costimulatory role of TL1A on TH1 polarized cells for IFN-γ production is consistent with its presence in IBD. TH1 polarization however has no effect on TL1A expression, indicating that TL1A expression in the mucosa is primarily maintained by TH17 or TH2 polarized cells. A role for TL1A has also been reported for experimental allergic encephalitis and for allergic lung inflammation in models for multiple sclerosis and asthma. In addition TL1A expression has been observed in the joints of rheumatoid arthritis patients, indicating similar regulation of inflammation by TL1A and TNFR25 in these diseases.

TNFR25 expression does not require new gene transcription but instead is rapidly upregulated within hours by mRNA splicing upon T cell activation through the PKC pathway. Antigen encounter by CD4 cells therefore will make TNFR25 available on the cell surface earlier than any other costimulatory TNF-receptor. Signaling of TNFR25 however is dependent on encountering the ligand TL1A. The sites and conditions for TL1A expression are not fully understood and require further study. However, early indications suggest that TL1A may be an important mediator of inflammation in the mucosal space including the airways and the digestive system.

### Example 2

### Immune Regulation, In Vitro or In Vivo

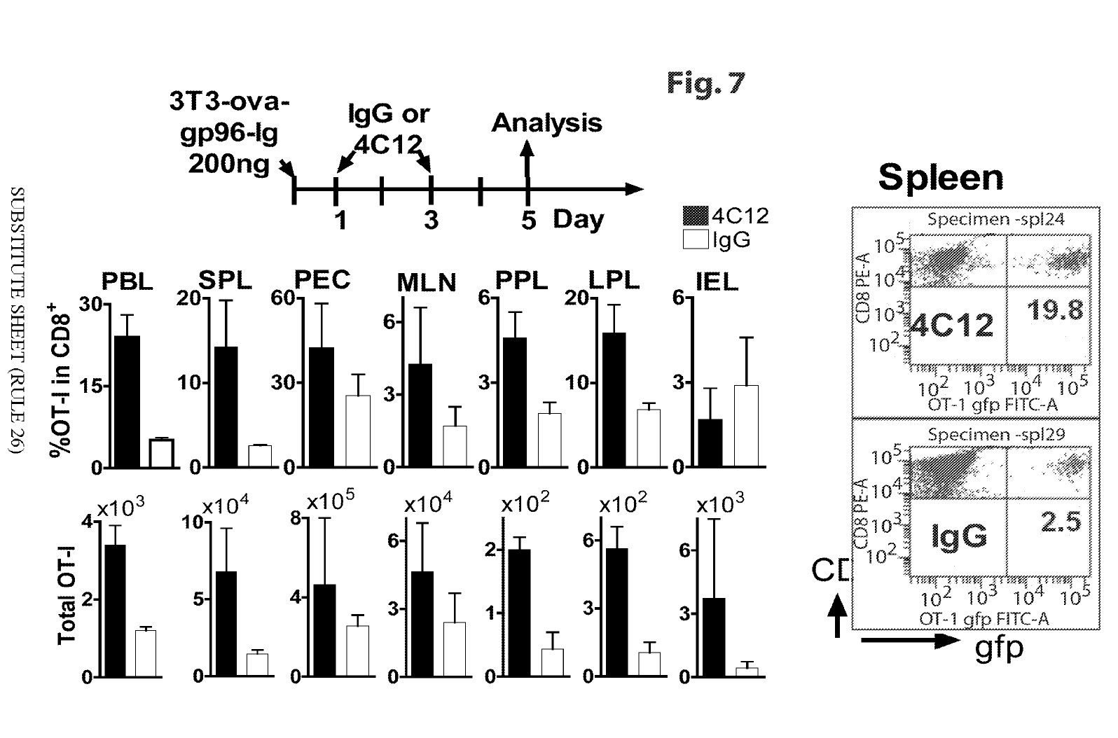

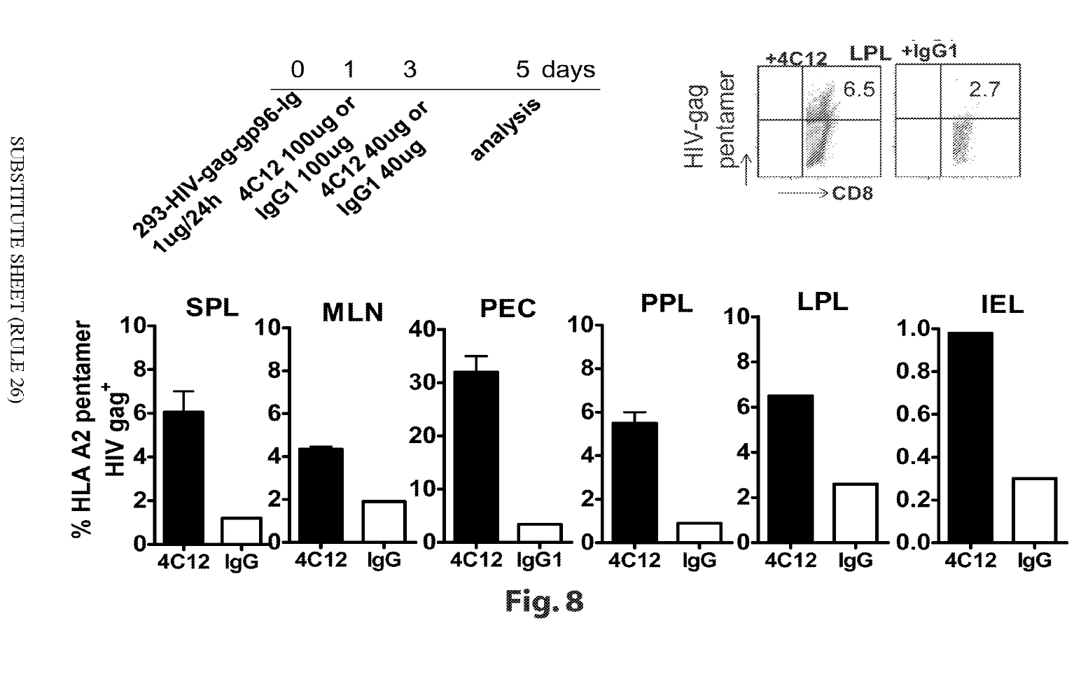

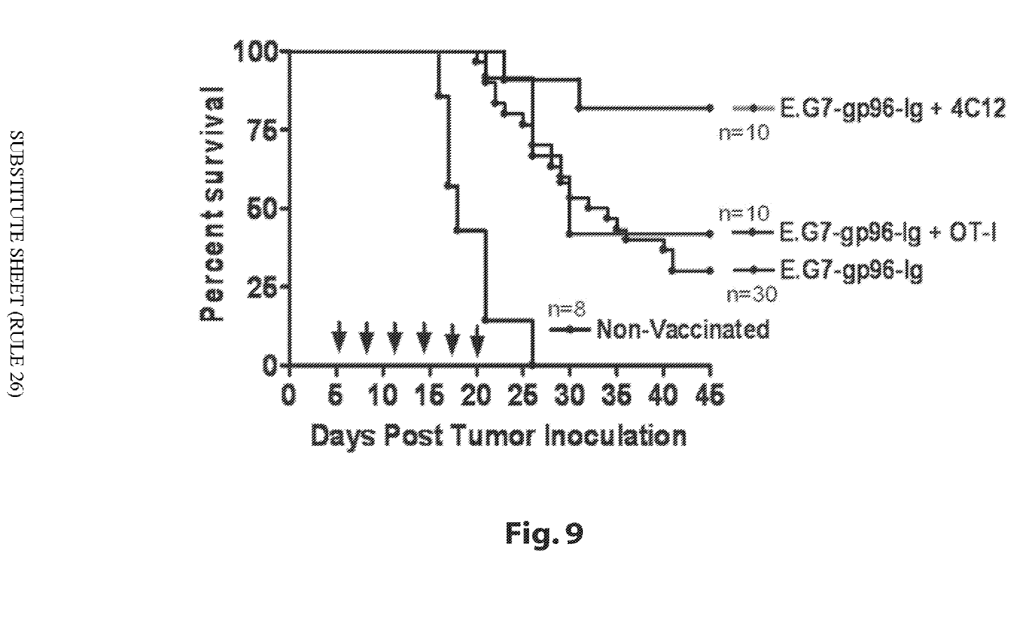

Cost and access of the second generation antiretroviral agents to the majority of those infected with HIV-1 disease underline the importance for the development of a therapeutic vaccine. To date efforts to develop a protective or therapeutic vaccine for HIV disease have fallen short owing in large part to our inability to measure immunologic correlates of efficacy. However, as new information emerges regarding regulatory elements of the immune response and anatomical compartments which impact on immune pathology, novel pathways for investigation are being explored. The results show that a novel cell based vaccine is capable of generating a vast augmentation in immunologic response to foreign antigen. See, the summary of results obtained in FIGS. 7 to 9. The goal of this project is to evaluate the safety and immune stimulatory effect of the gp96-HIV cell based vaccine.

Following work in the macaque, gp96-HIV vaccine will be evaluated in 15 HAART suppressed patients for safety and the generation of a cellular immune response. In addition to the generation of CTL against various epitopes, studies evaluating memory, activation, regulatory T-cells, and set point after treatment interruption will be evaluated. A second trial will involve the use of the gp96-HIV cell based vaccine with the added secretion and membrane product of ILIA, the cognate ligand to TNFR25 (DR3). TL1A is shown to suppress CD4 T-regulatory activity and enhance CD4 Th17 cells as defined through two phenotypic myeloid dendritic cell populations. As in the first trial, safety and the development of specific HIV cellular immunity will be evaluated. A third trial follows development, production, and testing in the macaque of an agonistic antibody to TNFR25. The distinction between this and the second trial is that TL IA is produced locally by the cell-based vaccine whereas the antibody is given separately and may have more systemic effects at the concentrations determined by the macaque studies. As with the other trials, safety and the development of specific HIV cellular immunity will be evaluated.

HIV/AIDS continues to infect and kill millions of people each year. The current medications are expensive and most of the affected people are unable to access them. The need for a vaccine to boost an infected individuals ability to fight infection is therefore important.

Although the invention has been illustrated and described with respect to one or more implementations, equivalent alterations and modifications will occur to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

The Abstract of the disclosure will allow the reader to quickly ascertain the nature of the technical disclosure. It is submitted with the understanding that it will not be used to interpret or limit the scope or meaning of the following claims.

What is claimed is:

1. A composition comprising mammalian TNFR25, TNFR25 splice variants, TNFR25-agonists, agonistic anti TNFR25, TNFR25 antagonists, TNFR25 ligands, other immune regulating molecules, variants, mutants or fragments thereof.

2. The composition of claim 2, wherein the agonists, antagonists or ligands comprise small molecules, ligands, antibodies, aptamers, organic compounds, inorganic compounds, nucleic acids or amino acids.

3. The composition of claim 1, wherein the mammalian TNFR25, TNFR25 splice variants, TNFR25-agonists, agonistic anti TNFR25, TNFR25 antagonists, TNFR25 ligands, other immune regulating molecules, variants, mutants or fragments thereof are human molecules.

4. The composition of claim 1, wherein a TNFR25 alternative splice variant lacks one or more domains or parts thereof as compared to wild type TNFR25 molecules.

5. The composition of claim 4, wherein the TNFR25 domains comprise, extracellular domains, intra-membrane domains or intra-cellular domains.

6. The composition of claim 4, wherein a TNFR25 alternative splice variant lacks one or more extracellular domains and/or intracellular domains, or combinations thereof.

7. A composition comprising mammalian TL1A, TL1A agonists, TL1A antagonists, TL1A ligands, other immune regulating molecules, ligands, variants, mutants or fragments thereof.

8. The composition of claim 7, wherein the agonists or antagonists comprise small molecules, ligands, antibodies, aptamers, organic compounds, inorganic compounds, nucleic acids or amino acids.

9. The composition of claim 7, wherein the mammalian TL1A, TL1A agonists, TL1A antagonists, TL1A ligands, other immune regulating molecules, ligands, variants, mutants or fragments thereof are human molecules.

10. A method of enhancing an immune response to a vaccine comprising:
administering to a patient in need thereof, a therapeutically effective amount of TNFR25, TNFR25 splice variants, TNFR25-agonists, agonistic anti TNFR25,TNFR25 antagonists, TNFR25 ligands, variants, mutants or fragments thereof, in conjunction, prior to or after administration of a vaccine or any combination thereof; and, enhancing an immune response to the vaccine.

11. The method of claim 10, wherein an antigen specific immune response is regulated.

12. The method of claim 10, wherein TL1A, TL1A agonists, TL1A antagonists, TL1A ligands, variants, mutants or fragments thereof are optionally administered.

13. The method of claim 10, wherein cytokines, cell factors are optionally administered as part of a treatment regimen.

14. A method of modulating an immune response to an antigen in a patient, comprising:
administering to the patient a therapeutically effective amount of at least one of: TNFR25, TNFR25 splice variants, TNFR25-agonists, agonistic anti TNFR25,TNFR25 antagonists, TNFR25 ligands, TL1A, TL1A ligands, TL1A agonists, TL1A antagonists, variants, mutants or fragments thereof; and,
modulating an immune response to an antigen in a patient.

15. The method of claim 14, wherein cytokines, growth factors, adjuvants, or combinations thereof are optionally administered to the patient.

16. The method of claim 14, wherein TL1A, TL1A agonists, TL1A antagonists, variants, mutants or fragments thereof are optionally administered.

17. The method of claim 14, wherein a patient can be a responder or non-responder to a particular antigen.

18. The method of claims 14, wherein the immune response comprises T cells (T lymphocytes), B cells (B lymphocytes), antigen presenting cells, dendritic cells, monocytes, macrophages, myeloid suppressor cells, natural killer (NK) cells, cytotoxic T lymphocytes (CTLs), inflammatory, or other infiltrates and subsets thereof, chemokines, cytokines, antibodies, factors, or hormones.

19. The method of claims 14, wherein one or more other immune regulatory molecules or ligands thereof are administered to the patient or cells.

20. A method of regulating a mucosal immune response in a patient, comprising:
administering to a patient, a therapeutically effective amount of at least one of: TNFR25, TNFR25 splice variants, TNFR25-agonists, agonistic anti TNFR25,TNFR25 antagonists, TNFR25 ligands, TL1A, TL1A ligands, TL1A agonists, TL1A antagonists, variants, mutants or fragments thereof, or any combination thereof; and,
regulating the mucosal immune response in the patient.

21. A method of regulating immune cell activity in vivo, comprising:
administering to a patient an effective amount of at least one of: TNFR25, TNFR25 splice variants, TNFR25-agonists, agonistic anti TNFR25,TNFR25 antagonists, TNFR25 ligands, TL1A, TL1A ligands, TL1A agonists, TL1A antagonists, variants, mutants or fragments thereof, or any combination thereof; and,
regulating immune cell activity in vivo.

22. The method of claim 21, wherein the immune cell activity is up-regulated or down regulated as compared to a normal control.

23. A method of regulating immune cells in vitro comprising culturing cells with at least one of: TNFR25, TNFR25-agonists, agonistic anti TNFR25, TNFR25 antagonists, TNFR25 ligands, TL1A, TL1A ligands, TL1A agonists, TL1A antagonists, variants, mutants or fragments thereof, or any combination thereof.

24. A method of polarizing a T lymphocyte helper (Th) response in vivo or in vitro, comprising:
administering to cells or a patient in need thereof, a therapeutically effective amount of:TNFR25, full length TNFR25, TNFR25 splice variants, TNFR25-agonists, agonistic anti TNFR25, TNFR25 antagonists, TNFR25 ligands, variants, mutants or fragments thereof, or any combination thereof; and,
polarizing a T lymphocyte helper (Th) response in vivo or in vitro.

25. The method of claim 22, wherein a T lymphocyte helper 2 (Th2) response is polarized to a T lymphocyte helper 1 (Th1) response.

26. The method of claim 25, further comprising administering interleukin-12 (IL-12), interferon gamma (IFN-γ) and anti-interleukin 4 (anti-IL-4) antibodies or blocking agents.

27. A method of treating a disease or disorder associated with an immune response comprising:
administering to a patient, a therapeutically effective amount of a composition comprising at least one of: TNFR25, TNFR25 splice variants, TNFR25-agonists, agonistic anti TNFR25, TNFR25 antagonists, TNFR25 ligands, TL1A, TL1A ligands, TL1A agonists, TL1A antagonists, variants, mutants or fragments thereof; and,
treating a disease or disorder associated with an immune response.

28. The method of claim 27, wherein a disease or disorder associated with an immune response comprising: autoimmunity, inflammation, allergies, asthma, colitis, multiple sclerosis, Crohn's disease, irritable bowel syndrome, or arthritis.

29. The method of claim 27, wherein the composition modulates T regulatory cell activity in vivo or in vitro.

30. An isolated cell expressing TNFR25 or TNFR25 splice variants.

31. An isolated nucleic acid encoding TNFR25 or TNFR25 splice variants.

32. A fusion protein comprising TNFR25, TNFR25 splice variants or fragments thereof.

33. An antibody or aptamer specific for TNFR25, TNFR25 splice variants or fragments thereof.
